(19) United States
(12) Patent Application Publication
(10) Pub. No.: US 2023/0225954 A1
(43) Pub. Date: Jul. 20, 2023

(54) TREATMENT OF MODERATE TO VERY SEVERE GLABELLAR LINES AND LATERAL CANTHAL LINES

(71) Applicants: Galderma Holding SA, Zug (CH); Ipsen Biopharm Limited, Wrexham (GB)

(72) Inventors: Eva AXEN, Järlåsa (SE); Xiaoming LIN, Keller, TX (US); Felipe WEINBERG, Stockholm (SE); Courtney MAGUIRE, Frisco, TX (US); Anna NILSSON, Uppsala (SE)

(21) Appl. No.: 18/096,899

(22) Filed: Jan. 13, 2023

Related U.S. Application Data
(60) Provisional application No. 63/399,127, filed on Aug. 18, 2022.
(60) Provisional application No. 63/299,705, filed on Jan. 14, 2022.

(51) Int. Cl. A61K 8/66; A61Q 19/08

(52) U.S. Cl. CPC A61K 8/66; C12Y 304/24069; A61Q 19/08

(57) ABSTRACT

Disclosed herein are methods of treatment of glabellar lines and lateral canthal lines using liquid botulinum neurotoxin compositions.

### CROSS-REFERENCE STATEMENT

This application claims priority under 35 U.S.C. § 119(e) to U.S. Provisional Application 63/299,705 filed Jan. 14, 2022, and U.S. Provisional Application 63/399,127 filed Aug. 18, 2022. The entire contents of these provisional applications are incorporated herein by reference.

### FIELD

Described herein are methods and compositions for treatment of moderate to severe and very severe glabellar lines (GL) and lateral canthal lines (LCL) using botulinum neurotoxin formulations. Also provided are liquid botulinum neurotoxin formulations and compositions.

### BACKGROUND

The following description of the background of the present technology is provided simply as an aid in understanding the present technology and is not admitted to describe or constitute prior art to the present technology.

Seven generally immunologically distinct botulinum neurotoxins have been characterized—botulinum neurotoxin serotypes A, B, C, D, E, F, and G—each of which is distinguishable by neutralization with type-specific antibodies. As one example, BOTOX® is the trademark of a botulinum toxin type A (BoNT-A) purified neurotoxin complex available commercially from Allergan, Inc. (Irvine, Calif.). BOTOX® is a popular injection-based cosmetic treatment that temporarily reduces the appearance of fine lines and wrinkles.

There are four BoNT-A products currently approved in the U.S.: BOTOX COSMETIC®, DYSPORT®, XEOMIN®, and JEUVEAU®. All of these products are stored in lyophilized or freeze-dried form for stability reasons. Despite each product containing a BoNT-A as the active ingredient, the activity, stability, and safety of each product is not the same. To the contrary, many of these products have comparatively variable activity, stability, and safety, all of which is compounded by the requirement of the aforementioned products to be reconstituted by a physician prior to administration.

Eyelid ptosis (“blepharoptosis”) has been documented as an adverse event in numerous BoNT-A clinical trials, with variable incidence in the treatment groups. Importantly, eyelid ptosis did not occur in any placebo groups, illustrating that it is directly attributable to botulinum toxin injection. In a review of studies with over 8000 patients in total, the rate of blepharoptosis was found to be about 2.5%. Specifically, eyelid ptosis incidences have been observed for Botox (3%), and Jeuveau (2%).

Thus, there is currently a need for a safe botulinum neurotoxin formulation that will not cause eyelid ptosis or cause less frequent and less severe eyelid ptosis, preferably in liquid form that is suitable for storage and use in therapy.

### SUMMARY OF THE INVENTION

The present disclosure is generally directed to compositions botulinum neurotoxin of and methods of treating lines and wrinkles such as lateral canthal lines (LCL), forehead lines and glabellar lines (GL).

In one aspect, the present disclosure provides methods of treating upper facial lines (e.g. glabellar lines (GL), lateral canthal lines (LCL), forehead lines, or a combination thereof) in a human subject with a reduced risk of eyelid ptosis, comprising administering a therapeutically effective amount of a liquid composition comprising a botulinum neurotoxin to the subject, thereby reducing the appearance of the GL, LCL, forehead lines, or combination thereof, wherein the botulinum neurotoxin is less likely to cause eyelid ptosis than a botulinum neurotoxin product with a lower enzymatic activity or a lower specific activity. In other words, there is provided a liquid composition comprising a botulinum neurotoxin for treating upper facial lines (e.g. glabellar lines (GL), lateral canthal lines (LCL), forehead lines, or a combination thereof) in a human subject with a reduced risk of eyelid ptosis, comprising administering a therapeutically effective amount of a liquid composition comprising a botulinum neurotoxin to the subject, thereby reducing the appearance of the upper facial lines (e.g. GL, LCL, forehead lines, or combination thereof), wherein the botulinum neurotoxin is less likely to cause eyelid ptosis than a botulinum neurotoxin product with a lower enzymatic activity or a lower specific activity. The term “enzymatic activity” may alternatively be referred to as “L-chain proteolytic activity” herein e.g. noting that the L-chain of a botulinum neurotoxin comprises a protease domain that cleaves target (SNARE) protein.

This observation is surprising because when a botulinum neurotoxin is employed for treating upper facial lines (as hereinbefore described), one would anticipate a botulinum neurotoxin having a relatively high potency (e.g. having a relatively high enzymatic activity, or having a relatively high specific activity) more likely to cause eyelid ptosis than a botulinum neurotoxin product having a relatively lower potency (e.g. having a relatively lower enzymatic activity or having a relatively lower specific activity). Eyelid ptosis occurs when a botulinum neurotoxin spreads away from the intended target muscles and into an adjacent eye lid muscle, causing flaccid paralysis (drooping) thereof. Thus, whilst a botulinum neurotoxin having a relatively high potency might be desirable for the purpose of achieving high cosmetic efficacy, one anticipated downside to such an approach is that said botulinum neurotoxin would be more likely to cause eye ptosis.

Another aspect provides a cosmetic method of treating upper facial lines (e.g. glabellar lines (GL), lateral canthal lines (LCL), forehead lines, or a combination thereof) in a human subject with a reduced risk of eyelid ptosis, comprising administering a therapeutically effective amount of a liquid composition comprising a botulinum neurotoxin to the subject, thereby reducing the appearance of the upper facial lines (e.g. GL, LCL, forehead lines, or combination thereof), wherein the botulinum neurotoxin is less likely to cause eyelid ptosis than a botulinum neurotoxin product with a lower enzymatic activity or a lower specific activity.

Another aspect provides a method of treating upper facial lines (e.g. GL, LCL, forehead lines, or combination thereof) of a subject, the method comprising administering a therapeutically effective amount of a botulinum neurotoxin (preferably a liquid composition comprising a botulinum neurotoxin) to the subject, wherein the severity of the facial lines is reduced, and wherein the subject experiences a reduced incidence of eyelid ptosis. Another aspect provides a method of treating upper facial lines (e.g. GL, LCL, forehead lines, or combination thereof) of a subject, the method comprising administering a therapeutically effective amount of a botulinum neurotoxin (preferably a liquid composition comprising a botulinum neurotoxin) to the subject, wherein the severity of the facial lines is reduced, and wherein the subject experiences a reduced incidence of eyelid ptosis when compared with incidence of eyelid ptosis in a subject treated with an alternative botulinum neurotoxin product having a lower enzymatic activity or a lower specific activity. In other words, there is provided a botulinum neurotoxin (preferably a liquid composition comprising a botulinum neurotoxin) for treating upper facial lines (e.g. GL, LCL, forehead lines, or combination thereof) of a subject, wherein the severity of the facial lines is reduced, and wherein the subject experiences a reduced incidence of eyelid ptosis. Another aspect provides a botulinum neurotoxin (preferably a liquid composition comprising a botulinum neurotoxin) for treating upper facial lines (e.g. GL, LCL, forehead lines, or combination thereof) of a subject, wherein the severity of the facial lines is reduced, and wherein the subject experiences a reduced incidence of eyelid ptosis when compared with incidence of eyelid ptosis in a subject treated with an alternative botulinum neurotoxin product having a lower enzymatic activity or a lower specific activity.

When treating glabellar lines, one typically administers botulinum neurotoxin at up to two sites of a corrugator muscle, and at one site of a procerus muscle.

When treating forehead lines, one typically administers botulinum neurotoxin at up to five sites of a frontalis muscle.

When treating lateral canthal lines, one typically administers botulinum neurotoxin and up to three sites at the external part of an orbicularis oculi muscle.

The upper facial lines may be glabellar lines. The facial lines may be moderate-to-severe facial lines, e.g. moderate facial lines or severe facial lines. The incidence of eyelid ptosis may less than 1%, preferably ≤0.9%. The total dose of botulinum neurotoxin administered (per visit/attendance/day) for treating glabellar lines may be 40-60 U (preferably 50 U), wherein 1 Unit is an amount of the botulinum neurotoxin corresponds to the calculated median lethal dose (LD50) in mice. The total dose of botulinum neurotoxin administered (per visit/attendance/day) for treating lateral canthal lines may be 50-70 U (preferably 60 U), wherein 1 Unit is an amount of the BoNT/A that corresponds to the calculated median lethal dose (LD50) in mice.

In some embodiments, the subject does not experience any serious adverse events. In some embodiments, the subject is less likely to experience eyelid ptosis compared to treatment with a botulinum neurotoxin product selected from Botox Cosmetic®, Xeomin®, and Jeuveau®. In some embodiments, the botulinum neurotoxin product with a lower enzymatic activity is selected from Botox Cosmetic®, Xeomin®, and Jeuveau®.

The botulinum neurotoxin may have an enzymatic activity normalized by concentration of botulinum neurotoxin of about 1.16 BU/BoNT. Additionally or alternatively, the present disclosure provides a method of treating glabellar lines (GL), lateral canthal lines (LCL), forehead lines, or a combination thereof with a reduced risk of eyelid ptosis in a human subject in need thereof, comprising administering a therapeutically effective amount of a liquid composition comprising a botulinum neurotoxin to the subject, thereby reducing the appearance of the GL, LCL, forehead lines, or combination thereof, wherein the botulinum neurotoxin has an enzymatic activity normalized by concentration of botulinum neurotoxin of about 1.16 BU/BoNT. The human subject may be at risk of developing or considered likely to develop eyelid ptosis upon the administration of botulinum neurotoxin (e.g., a botulinum neurotoxin formulation with an enzymatic activity normalized by concentration of botulinum neurotoxin of less than 1.16 BU/BoNT or a specific activity of less than about 2.0×10⁸ U/mg, such as Botox Cosmetic®, Xeomin® and Jeuveau®). The human subject may also be suffering from eyelid ptosis and/or have a history of developing eyelid ptosis upon the administration of botulinum neurotoxin. In another embodiment, the human subject is not one that (i) has no at risk of developing eyelid ptosis, (ii) is not suffering from eyelid ptosis and/or, (iii) has no history of developing eyelid ptosis upon the administration of botulinum neurotoxin at any dose or enzymatic activity.

The liquid composition may possess an activity of about 97 BU/ml or greater, and, optionally, the liquid composition may possess an enzymatic activity normalized by concentration of the botulinum neurotoxin of about 10. In another aspect, the present disclosure provides methods of treating glabellar lines (GL), lateral canthal lines (LCL), forehead lines, or a combination thereof in a human subject, comprising administering a therapeutically effective amount of a liquid composition comprising a botulinum neurotoxin to the subject, thereby reducing the appearance of the GL, LCL, forehead lines, or combination thereof, wherein (i) the liquid composition possesses an activity of about 97 BU/ml or greater, and, optionally, the liquid composition possesses an enzymatic activity normalized by concentration of the botulinum neurotoxin of about 10.

In some embodiments of any of the foregoing methods, the liquid formulation has a specific BoNT activity of about 2.0×10⁸ U/mg total protein, wherein the specific activity (U/mg) is optionally measured using a mouse LD50 potency as U/ml divided by the total amount of protein as determined by a μBCA method (mg/ml).

In some embodiments of any of the foregoing methods, the liquid formulation has an enzymatic activity normalized by concentration of botulinum neurotoxin of about 1.16 BU/BoNT, wherein the enzymatic activity of the botulinum neurotoxin is optionally determined using a BOTEST™.

In some embodiments of any of the foregoing methods, the liquid formulation has relative potency normalized by concentration of botulinum neurotoxin of about 1.20 units/BoNT, wherein the enzymatic activity of the botulinum neurotoxin is determined using a cell-based assay.

In some embodiments of any of the foregoing methods, the liquid formulation contains no human- or animal-derived excipients.

In some embodiments of any of the foregoing methods, the occurrence of eyelid ptosis may be less than 2%. A percentage of eyelid ptosis as referred to herein preferably corresponds to the percentage of a patients in a cohort (e.g. a cohort of at least 50 patients) that demonstrate eyelid ptosis, wherein each patient of the cohort is administered the BoNT formulation. This means that the, when a liquid composition described herein is administered to a cohort of subject, less than 2% of said cohort will experience eyelid ptosis. In some embodiments of any of the foregoing methods, the occurrence of eyelid ptosis is less than 2%, less than 1.9%, less than 1.8%, less than 1.7%, less than 1.5%, less than 1.4%, less than 1.3%, less than 1.2%, less than 1.1%, less than 1.0%, or as low as 0.9%. For example, the occurrence of eyelid ptosis may be ≤1.0%. Preferably, the occurrence of eyelid ptosis may be ≤0.9%. Thus, the occurrence of eyelid ptosis following treatment with an alternative botulinum neurotoxin product (e.g. botulinum neurotoxin product with a lower enzymatic activity or a lower specific activity) may be greater than 2%.

In some embodiments of any of the foregoing methods, the subject does not experience eyelid ptosis.

In some embodiments of any of the foregoing methods, the subject is less likely to experience eyelid ptosis compared to treatment with a botulinum neurotoxin product selected from Botox Cosmetic®, Xeomin®, and Jeuveau®. In some embodiments of any of the foregoing methods, the subject is less likely to experience eyelid ptosis compared to treatment with an alternative botulinum neurotoxin product that was stored in a dry form (e.g. lyophilized powder form) and reconstituted for administration to the subject.

In some embodiments of any of the foregoing methods, the GL, LCL, or combination thereof are moderate to severe.

In some embodiments of any of the foregoing methods, the GL, LCL, or combination thereof are severe or highly severe.

In some embodiments of any of the foregoing methods, the liquid composition comprising the botulinum neurotoxin does not comprise any animal proteins or companion proteins.

In some embodiments of any of the foregoing methods, the subject is treated only for GL. In some embodiments of any of the foregoing methods, the subject is treated only for LCL. In some embodiments of any of the foregoing methods, the subject in treated for a combination of GL and LCL. In any of these embodiments, the GL and/or LCL may be on one or both sides of the subject's face.

In some embodiments of any of the foregoing methods, the treatment provides a response rate that is higher than Botox Cosmetic®. In some embodiments of any of the foregoing methods, the treatment provides a response rate that is higher than an alternative botulinum neurotoxin product that was stored in a dry form (e.g. lyophilized powder form) and reconstituted for administration to the subject.

In some embodiments of any of the foregoing methods, the liquid composition comprises about 4 buffering agents. For example, the liquid composition can comprise a first buffering agent present at a concentration of about 100 to about 300 mM, or at a concentration of about 0.1-10 mg/mL. The liquid composition can comprise a second buffering agent present at a concentration of about 1 to about 25 mM, or at a concentration of about 0.1-1.0 mg/mL. The liquid composition can comprise a third buffering agent present at a concentration of about 1 to about 25 mM, or at a concentration of about 0.1-1.0 mg/mL. The liquid composition can comprise a fourth buffering agent present at a concentration of about 1 to about 25 mM, or at a concentration of about 0.1-1.0 mg/mL. The liquid composition can comprise a fifth buffering agent present at a concentration of about 1 to about 25 mM, or at a concentration of about 0.1-1.0 mg/mL. In some embodiments, the buffering agents are selected from the group consisting of sodium chloride, potassium chloride, sodium phosphate, potassium phosphate, di-sodium hydrogen phosphate dihydrate, and sodium dihydrogen phosphate dihydrate.

In some embodiments of any of the foregoing methods, the liquid composition comprises about one stabilizer, which is, optionally, an amino acid. For example, the amino acid can be selected from the group consisting of alanine, valine, leucine, isoleucine, methionine, phenylalanine, tyrosine, and tryptophan. In some embodiments, the amino acid is in the D isoform or the L isoform. In some embodiments, the amino acid is present at a concentration of about 0.1 to about 3.0 mg/mL.

In some embodiments of any of the foregoing methods, the liquid composition comprises about one surfactant, which is, optionally, a non-ionic surfactant. In some embodiments, the non-ionic surfactant is present at a concentration of about 0.01% (v/v) to about 5.0% (v/v), or at a concentration of about 0.1 to about 3.0 mg/mL.

In some embodiments of any of the foregoing methods, the botulinum neurotoxin is selected from the group consisting of botulinum neurotoxin types A, B, C, D, E, F, and G. In particular, the botulinum neurotoxin can be botulinum neurotoxin type A.

In some embodiments of any of the foregoing methods, the pH of the liquid composition is between 6.6 and 6.9. In some embodiments, the pH can be 6.75 or about 6.75.

In some embodiments of any of the foregoing methods, the botulinum neurotoxin has a molecular weight of about 150 kDa.

In some embodiments of any of the foregoing methods, the osmolality of the liquid composition is between 270 mosm/kg and 310 mosm/kg.

In some embodiments of any of the foregoing methods, between 1 and 100 units of botulinum toxin is administered to the subject. In some embodiments, between 10 and 75 units of botulinum toxin is administered to the subject. In some embodiments, between 25 and 75 units of botulinum toxin is administered to the subject. In some embodiments, 10, 25, 30, 45, 50, 60, 75, or 90 units of botulinum toxin is administered to the subject.

In some embodiments of any of the foregoing methods, the liquid composition is administered by injection. The injection can be, for example, subdermal, transdermal, intradermal or intramuscular. In some embodiments, the subject is injected multiple times in the glabellar region. In some embodiments, adjacent injections are separated by about 0.5 to about 10 cm. In some embodiments, adjacent injections are separated by about 1.5 to about 3 cm. In some embodiments, the injections are in the procerus muscle and the corrugator supercillii muscles on one or both sides of the face. In some embodiments, the injections are first made in the procerus muscle followed by the corrugator supercillii muscles on one or both sides of the face, moving outwards from the median. In some embodiments, all the injections are about 1 cm above the upper orbital rim and internal to the mid-pupillary lines. In some embodiments, all the injections are about 1 cm above the central eyebrow or the bony supraorbital ridge. In some embodiments, the subject is injected multiple times below the lateral canthus, in the external part of the orbicularis oculi, and/or 1-2 cm from the orbital rim. In some embodiments, the subject is further injected multiple times below the lateral canthus, in the external part of the orbicularis oculi, and/or 1-2 cm from the orbital rim.

In some embodiments of any of the foregoing methods, the method is repeated at intervals from about 1 month to about 6 months to inhibit recurrence of GL, LCL, or a combination thereof. In some embodiments, the method is repeated at intervals from about 3 months to about 6 months to inhibit such recurrence. In some embodiments, the method is repeated at intervals of about 4 months to inhibit such recurrence.

The present disclosure also provides liquid compositions comprising a botulinum neurotoxin for use in treating glabellar lines and/or lateral canthal lines in a subject as disclosed herein.

The present disclosure also provides uses of liquid compositions comprising a botulinum neurotoxin for treating glabellar lines and/or lateral canthal lines as disclosed herein.

The following detailed description is exemplary and explanatory, but it is not intended to be limiting.

### BRIEF DESCRIPTION OF THE DRAWINGS

FIG. 1 shows MAS GL Scale at Rest and MAS GL Scale Dynamic.

FIG. 2 shows the glabellar line severity scoring matrix.

FIG. 3 shows the results of the ILA responder rate for the subjects of the glabellar lines QM1114-DP study over time.

FIG. 4 shows Global Aesthetic Improvement Score (GAIS) analysis of the subjects of the glabellar lines QM1114-DP study over time.

FIG. 5 shows safety results (related adverse events) of the glabellar lines QM1114-DP study.

FIG. 6 shows the lateral canthal line severity scoring matrix.

FIG. 7 shows the results of the ILA responder rate for the subjects of the lateral canthal lines QM1114-DP study over time.

FIG. 8 shows Global Aesthetic Improvement Score (GAIS) analysis of the subjects of the lateral canthal lines QM1114-DP study over time.

FIG. 9 shows safety results (related adverse events) of the lateral canthal lines QM1114-DP study.

FIG. 10 shows the results of the ILA responder rate for glabellar lines over time.

FIG. 11 shows the results of the ILA responder rate for lateral canthal lines over time.

FIG. 12 shows Global Aesthetic Improvement Score (GAIS) analysis.

FIG. 13 shows composite 2-grade improvement for glabellar lines.

FIG. 14 shows Investigator Score for glabellar lines.

FIG. 15 composite 2-grade improvement for lateral canthal lines.

FIG. 16 shows Investigator Score for lateral canthal lines.

FIG. 17 shows enzymatic activity measured using BoTest™.

FIG. 18 shows specific activity measured using a BoTest™ normalized by BoNT-A specific ELISA (BOLISA™). The top panel shows the results from a single repeat of a specific activity experiment with the units being BoTest U/RBU. The bottom panel shows U/pg.

FIG. 19 shows relative potency from a cell-based assay normalized by concentration of BoNT-A.

FIG. 20 shows a formulation (QM-1114) with a high level of purity (over 98%) in the main peak of UPLC-SEC.

FIG. 21 shows results from SDS-PAGE analysis in which no detectable impurities were observed, but only the expected bands from BoNT-A1.

FIG. 22 shows month 1 LCL composite 2-grade responders based on ILA and SLA assessments at maximum smile. 26 subjects treated with QM-1114 and 4 subjects treated with placebo had Month 1 assessments conducted remotely and were not included in this analysis. CMH, Cochran-Mantel-Haenszel; ILA, investigator live assessment; LCL, lateral canthal lines; SLA, subject live assessment.

FIG. 23 shows time onset of treatment effect. Specifically, the time to onset of LCL treatment based on subject diary.

FIG. 24 shows responder rates based on investigator assessments at maximum smile.

FIG. 25 shows investigator-reported responder rates at maximum smile were ≥1-grade improvement in LCL severity.

FIG. 26 shows time for lateral canthal lines (LCL) to return to baseline score following treatment.

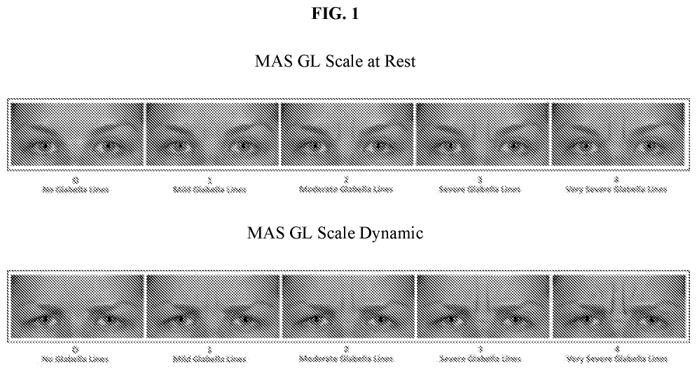

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless the context indicates otherwise, it is specifically intended that the various features of the methods described herein can be used in any combination. Moreover, the disclosure also contemplates that in some embodiments, any feature or combination of features set forth herein can be excluded or omitted. To illustrate, if the specification states that a method comprises steps A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### I. Definitions

As used herein, the singular forms “a,” “an,” and “the” designate both the singular and the plural, unless expressly stated to designate the singular only.

It is to be understood, although not always explicitly stated, that all numerical designations are preceded by the term “about.” The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 15%, 10%, 5%, 1%, or 0.1% of the particular term.

Also as used herein, “and/or” refers to and encompasses any and all possible combinations of one or more of the associated listed items, as well as the lack of combinations when interpreted in the alternative (“or”).

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject. Administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule. The pharmaceutical compositions disclosed herein are “locally administered” (local administration), that is administered at or in the vicinity of the site at which a therapeutic result or outcome is desired. Administration can be unilateral or bilateral, as needed for a given patient.

The terms “treat”, “treating” or “treatment”, as used herein, include reducing or improving the appearance of glabellar lines (GL) and/or lateral canthal lines (LCL), or one or more symptoms thereof, whether or not GL or LCL are considered to be “cured” or “eliminated” and whether or not all symptoms are resolved. The terms also include reducing or preventing progression of GL, LCL, and/or one or more symptoms thereof, and achieving any therapeutic and/or prophylactic benefit.

The term “Botulinum toxin” means a botulinum neurotoxin type A, B, C, D, E, F or G as either pure toxin (i.e. the about 150 kiloDalton molecular weight neurotoxic component) or as a botulinum toxin complex (about 300 to about 900 kiloDalton molecular weight), including recombinant, chimeric, hybrid, retargeted, and amino acid sequence modified botulinum neurotoxins, but excluding botulinum toxins which are not neurotoxins such as the cytotoxic botulinum toxins C₂ and C₃.

“Local administration” means administration (i.e. by a subcutaneous, intramuscular, subdermal, intradermal, subcutaneous, intra-organ, e.g. injected into the bladder wall or into the body of the prostate] or transdermal route) of a pharmaceutical agent to or to the vicinity of a target tissue, muscle or subdermal location by a non-systemic route. Thus, local administration excludes systemic (i.e. to the blood circulation system) routes of administration, such as intravenous or oral administration. Peripheral administration means administration to the periphery (i.e. to a location on or within a face, limb, trunk or head of a patient) as opposed to a visceral or gut (i.e. to the viscera) administration.

“Pharmaceutical composition” means a formulation in which an active ingredient (the active agent) can be a botulinum neurotoxin. The word “formulation” means that there is at least one additional ingredient in the pharmaceutical composition besides the active agent. A pharmaceutical composition is therefore a formulation that is suitable for cosmetic, diagnostic, or therapeutic administration (i.e. by subdermal or intramuscular injection) to a subject, such as a human patient.

An “effective amount” is an amount sufficient to effect beneficial or desired results such as lessening, smoothing, or straightening glabellar lines and/or lateral canthal lines. An effective amount as used herein would also include an amount sufficient prevent worsening of glabellar line or lateral canthal line formation, or reverse the formation of existing glabellar lines. Thus, it is not possible to specify the exact “effective amount.” An effective amount can be administered in one or more administrations, applications, or dosages. Such delivery is dependent on a number of variables including the time period for which the individual dosage unit is to be used, the location of administration of the agent, the severity of the glabellar lines, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. The dosage can be determined by a physician and adjusted, as necessary, to suit observed effects of the treatment. Preferable, the effective amount does not cause side effects (e.g., eyelid ptosis).

### II. Liquid Composition of BoNT

Provided herein are liquid compositions comprising a botulinum neurotoxin (BoNT) and a buffer that are suitable for storage as a liquid and for treatment of glabellar lines without further reconstitution or admixing. In other words, the disclosed liquid compositions are “ready-to-use” and do not require any sort of specialized preparation, unlike other BoNT products that are currently on the market. Thus, a liquid composition of the present invention may be contrasted with an alternative botulinum neurotoxin product that is stored in a dry form (e.g. lyophilized powder form) and requires reconstitution for administration to a subject.

The botulinum neurotoxin (BoNT) is a protein dimer of 150 kilodalton (kDa) consisting of a 100 kDa heavy chain and 50 kDa light chain. The two chains are connected by a disulphide bond of two cysteine residues. The light chain is an enzyme that cuts the synaptosomal-associated protein of 25 kDa (SNAP-25). The heavy chain mediates binding and internalization of the toxin protein. Unlike other commercially available BoNTs, the botulinum neurotoxin in the present liquid composition (an exemplary embodiment of which is herein referred to as QM1114) is stable in liquid form and does not require reconstitution or admixing prior to use. In some embodiments, the BoNT formulated in the liquid composition is botulinum neurotoxin type A (BoNT-A1). As outlined in the Examples section, an exemplary embodiment of the present liquid composition was investigated in human clinical trials, and is referred to as QM1114 (or QM1114-DP). Thus, QM1114 is a representative embodiment of the liquid composition of the invention which will now be described in more detail with reference to its excipients and properties.

Moreover, for the purposes of this disclosure, the liquid composition (e.g., QM1114) does not contain any human or animal-derived excipients, and such a formulation can be manufactured using an entirely animal-free process at a high level of purity using filtration and/or chromatography. The process can be free from precipitation steps or freeze drying. The formulation may have a neutral or nearly neutral pH (e.g., a pH of 6.5, 6.6, 6.7, 6.75, 6.8, 6.9, 7.0, 7.1, 7.2, 7.3, 7.4, 7.5). In some embodiments, the pH may be about 6.75.

The specific activity of the liquid composition (e.g., QM1114) is as high or higher than competitive products. Specific activity may refer to how active a certain amount of the toxin is, and can be established by dividing a measure of activity (e.g., a cell-based potency assay or a BOTEST™) by a measure of the amount of enzyme present during the measure of activity (e.g., a BOLISA®). For example, the specific BoNT activity may be about 1.5×10⁸, about 1.6×10⁸, about 1.7×10⁸, about 1.8×10⁸, about 1.9×10⁸, about 2.0×10⁸, about 2.1×10⁸, about 2.2×10⁸, about 2.3×10⁸, about 2.4×10⁸, or about 2.5×10⁸ U/mg total protein or more, wherein the specific activity (U/mg) is measured using a mouse LD50 potency as U/ml divided by the total amount of protein as determined by a μBCA method (mg/ml). In some embodiments, the specific activity may be about 2.0×10⁸ U/mg total protein. In some embodiments, the disclosed liquid composition (e.g., QM1114) has a higher specific activity than other clinical BoNT formulations such as Botox Cosmetic®, Xeomin® and Jeuveau®. In some embodiments, the disclosed liquid composition (e.g., QM1114) has a higher specific activity than other clinical BoNT formulations that are stored in a dry form (e.g. lyophilized powder form) and require reconstitution for administration to a subject.

The liquid composition (e.g., QM1114) can comprise a buffer, which comprises sodium, chloride, and/or phosphate ions. The addition of such ions usually occurs through the addition of buffering salts. In some embodiments, the formulation may comprise a saline phosphate buffer, and in some embodiments there may be no human- or animal-derived excipients.

For example, the liquid composition (e.g., QM1114) may comprise at least one source of chloride ions, such as sodium chloride, potassium chloride, or another source of chloride ions at a concentration of about 100 to about 300 mM, such as 100, 110, 120, 130, 140, 150, 160, 170, 180, 190, 200, 210, 220, 230, 240, 250, 260, 270, 280, 290, or 300 mM, or at a concentration of about 1 to about 25 mM, such as 1, 2, 3, 4, 5, 6, 7, 8, 9, 10, 11, 12, 13, 14, 15, 16, 17, 18, 19, 20, 21, 22, 23, 24, 25 mM. In some embodiments, the liquid composition (e.g., QM1114) may comprise more than one source of chloride ions at the same or differing concentrations, for instance, sodium chloride or another source of sodium or chloride ions at a concentration of about 100 to about 300 mM, such as 100, 110, 120, 130, 140, 150, 160, 170, 180, 190, 200, 210, 220, 230, 240, 250, 260, 270, 280, 290, or 300 mM, and potassium chloride or another source of chloride ions at a concentration of about 1 to about 25 mM, such as 1, 2, 3, 4, 5, 6, 7, 8, 9, 10, 11, 12, 13, 14, 15, 16, 17, 18, 19, 20, 21, 22, 23, 24, 25 mM. In some embodiments, the one or more source(s) of sodium/chloride ions may be present at the same or differing concentrations in the range of about 0.1-10 mg/mL, such as 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 mg/mL or any value in between.

Additionally or alternatively, the liquid composition (e.g., QM1114) may comprise at least one source of phosphate ions, such as sodium phosphate, potassium phosphate, di-sodium hydrogen phosphate dihydrate, sodium dihydrogen phosphate dihydrate, or another source of phosphate ions at a concentration of about 1 to about 50 mM or about 5 to about 15 mM, such as 1, 5, 10, 15, 20, 25, 30, 35, 40, 45, or 50 mM. In some embodiments, the liquid composition may comprise more than one source of phosphate ions at the same or differing concentrations, for instance, sodium phosphate, potassium phosphate, di-sodium hydrogen phosphate dihydrate, sodium dihydrogen phosphate dihydrate, or another source of phosphate ions at a concentration of about 1 to about 50 mM or about 5 to about 15 mM, such as 1, 5, 10, 15, 20, 25, 30, 35, 40, 45, or 50 mM, and a different source of phosphate ions selected from sodium phosphate, potassium phosphate, di-sodium hydrogen phosphate dihydrate, sodium dihydrogen phosphate dihydrate, or another source of phosphate ions at a concentration of about 1 to about 50 mM or about 5 to about 15 mM, such as 1, 5, 10, 15, 20, 25, 30, 35, 40, 45, or 50 mM. In some embodiments, the one or more source(s) of phosphate ions may be present at the same or differing concentrations in the range of about 0.1-1.0 mg/mL, such as 0.1, 0.2, 0.3, 0.4, 0.5, 0.6, 0.7, 0.8, 0.9, or 1.0 mg/mL or any value in between.

In some embodiments, the liquid composition (e.g., QM1114) may comprise 1-5 or more buffering agents. Thus, the liquid composition (e.g., QM1114) may comprise 1, 2, 3, 4, or 5 or more buffering agents, including but not limited to, sodium chloride, potassium chloride, sodium phosphate, potassium phosphate, di-sodium hydrogen phosphate dihydrate, or sodium dihydrogen phosphate dihydrate. The 1, 2, 3, 4, or 5 or more buffering agents may be present at the same or differing concentrations. For example, in some embodiments, a first buffering agent (e.g., sodium chloride, potassium chloride, sodium phosphate, potassium phosphate, di-sodium hydrogen phosphate dihydrate, or sodium dihydrogen phosphate dihydrate) may be present at a concentration of about 100 to about 300 mM, such as 100, 110, 120, 130, 140, 150, 160, 170, 180, 190, 200, 210, 220, 230, 240, 250, 260, 270, 280, 290, or 300 mM, or at a concentration of about 0.1-10 mg/mL, such as 0.1, 0.2, 0.3, 0.4, 0.5, 0.6, 0.7, 0.8, 0.9, 1.0, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 mg/mL or any value in between. In some embodiments, a second buffering agent (e.g., sodium chloride, potassium chloride, sodium phosphate, potassium phosphate, di-sodium hydrogen phosphate dihydrate, or sodium dihydrogen phosphate dihydrate) may be present at a concentration of about 1 to about 25 mM, such as 1, 2, 3, 4, 5, 6, 7, 8, 9, 10, 11, 12, 13, 14, 15, 16, 17, 18, 19, 20, 21, 22, 23, 24, 25 mM, or at a concentration of about 0.1-1.0 mg/mL, such as 0.1, 0.2, 0.3, 0.4, 0.5, 0.6, 0.7, 0.8, 0.9, or 1.0 mg/mL or any value in between. In some embodiments, a third buffering agent (e.g., sodium chloride, potassium chloride, sodium phosphate, potassium phosphate, di-sodium hydrogen phosphate dihydrate, or sodium dihydrogen phosphate dihydrate) may be present at a concentration of about 1 to about 25 mM, such as 1, 2, 3, 4, 5, 6, 7, 8, 9, 10, 11, 12, 13, 14, 15, 16, 17, 18, 19, 20, 21, 22, 23, 24, 25 mM, or at a concentration of about 0.1-1.0 mg/mL, such as 0.1, 0.2, 0.3, 0.4, 0.5, 0.6, 0.7, 0.8, 0.9, or 1.0 mg/mL or any value in between. In some embodiments, a fourth buffering agent (e.g., sodium chloride, potassium chloride, sodium phosphate, potassium phosphate, di-sodium hydrogen phosphate dihydrate, or sodium dihydrogen phosphate dihydrate) may be present at a concentration of about 1 to about 25 mM, such as 1, 2, 3, 4, 5, 6, 7, 8, 9, 10, 11, 12, 13, 14, 15, 16, 17, 18, 19, 20, 21, 22, 23, 24, 25 mM, or at a concentration of about 0.1-1.0 mg/mL, such as 0.1, 0.2, 0.3, 0.4, 0.5, 0.6, 0.7, 0.8, 0.9, or 1.0 mg/mL or any value in between. In some embodiments, a fifth buffering agent (e.g., sodium chloride, potassium chloride, sodium phosphate, potassium phosphate, di-sodium hydrogen phosphate dihydrate, or sodium dihydrogen phosphate dihydrate) may be present at a concentration of about 1 to about 25 mM, such as 1, 2, 3, 4, 5, 6, 7, 8, 9, 10, 11, 12, 13, 14, 15, 16, 17, 18, 19, 20, 21, 22, 23, 24, 25 mM, or at a concentration of about 0.1-1.0 mg/mL, such as 0.1, 0.2, 0.3, 0.4, 0.5, 0.6, 0.7, 0.8, 0.9, or 1.0 mg/mL or any value in between.

Other components may be included in the liquid composition in order to improve stability or other properties of the composition. For example, applicable stabilizers may include, but are not limited to amino acids (e.g., alanine, valine, leucine, serine, threonine, lysine histidine, tryptophan, aspartic acid, or glutamic acid), sodium hydrogen sulfite, sodium citrate or other citrates, etc. In some embodiments, the amino acid may be an amino acid with a hydrophobic side chain (e.g., alanine, valine, leucine, isoleucine, methionine, phenylalanine, tyrosine, and tryptophan). In some embodiments, the amino acid may be in the D isoform, while in some embodiments, the amino acid may be in the L isoform. Thus, in some embodiments, the liquid composition may comprise at least one D- or L-amino acid (e.g., alanine, valine, leucine, serine, threonine, lysine histidine, tryptophan, aspartic acid, or glutamic acid) at a concentration of about 0.1 to about 3.0 mg/mL, about 0.5 to about 2.5 mg/mL, or about 0.75 to about 2.25 mg/mL. In some embodiments, the liquid composition may comprise at least one D- or L-amino acid (e.g., alanine, valine, leucine, serine, threonine, lysine histidine, tryptophan, aspartic acid, or glutamic acid) at a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mg/mL or any value in between. In some embodiments, the liquid composition may comprise at least one D- or L-amino acid (e.g., alanine, valine, leucine, serine, threonine, lysine histidine, tryptophan, aspartic acid, or glutamic acid) at a concentration of about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 mg/mL or any value in between.

In some embodiments, the liquid composition (e.g., QM1114) may further comprise one or more surfactants (e.g., non-ionic surfactants like a polysorbate (e.g., polysorbate 80 or polysorbate 20) or nonoxynols; anionic surfactants like docusate; or cationic surfactants like quaternary ammonium salts). Thus, in some embodiments, the liquid composition may comprise a non-ionic surfactant, including but not limited to a polysorbate (e.g., polysorbate 80 or polysorbate 20) or a nonoxynol. In some embodiments, the liquid composition may comprise an anionic surfactant, including but not limited to docusate. In some embodiments, the liquid composition may comprise a cationic surfactant, including but not limited to a quaternary ammonium salt. In some embodiments, the surfactant may be present in a concentration of about 0.01% (v/v) to about 5.0% (v/v), about 0.05% (v/v) to about 2.5% (v/v), or about 0.1% (v/v) to about 1.5% (v/v). In some embodiments, the at least one surfactant may be present in a concentration of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0% (v/v) or any value in between. In some embodiments, the at least one surfactant may be present in a concentration of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, or about 5.0% (v/v) or any value in between. In some embodiments, the liquid composition may comprise at least one surfactant at a concentration of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mg/mL or any value in between. In some embodiments, the liquid composition may comprise at least one surfactant at a concentration of about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0 mg/mL or any value in between.

In some embodiments, the liquid composition may further comprise one or more emulsifiers (e.g., soy lecithin), wetting agents, excipients (lactose, mannitol, glucose, microcrystal cellulose, colloidal silica, and starch, etc.), binders (hydroxypropylcellulose, polyvinylpyrrolidone, and magnesium metasilicate aluminate, etc.), disintegrator (starch, L-hydroxypropylcellulose, carboxymethylcellulose, crosscarmellose sodium, and cellulose calcium glycolate, etc.), lubricants (magnesium stearate etc.), swelling agents (hydroxypropylcellulose, hydroxypropylmethylcellulose, carbopole, carboxymethylcellulose, polyvinyl alcohol, xanthan gum, and Cyamoposis Gum, etc.), swelling adjuvants (glucose, fructose, mannitol, xylitol, erythritol, maltose, trehalose, phosphate salt, citrate, silicate, glycine, glutamate, and arginine, etc.), and/or solubilizers (polyethylene glycol, propylene glycol, etc.).

In some embodiments, the liquid composition may comprise 1-5 or more buffering agents (e.g., sodium chloride, potassium chloride, sodium phosphate, potassium phosphate, di-sodium hydrogen phosphate dihydrate, or sodium dihydrogen phosphate dihydrate); one or more stabilizers (e.g., alanine, valine, leucine, serine, threonine, lysine histidine, tryptophan, aspartic acid, or glutamic acid); and one or more surfactants (e.g., non-ionic surfactants like a polysorbate (e.g., polysorbate 80 or polysorbate 20) or nonoxynols; anionic surfactants like docusate; or cationic surfactants like quaternary ammonium salts). In some embodiments, the liquid composition may comprise: (i) a first buffering agent (e.g., sodium chloride, potassium chloride, sodium phosphate, potassium phosphate, di-sodium hydrogen phosphate dihydrate, or sodium dihydrogen phosphate dihydrate) at a concentration of about 100 to about 300 mM, or at a concentration of about 0.1-10 mg/mL; (ii) a second buffering agent (e.g., sodium chloride, potassium chloride, sodium phosphate, potassium phosphate, di-sodium hydrogen phosphate dihydrate, or sodium dihydrogen phosphate dihydrate) at a concentration of about 1 to about 25 mM, or at a concentration of about 0.1-1.0 mg/mL; (iii) a third buffering agent (e.g., sodium chloride, potassium chloride, sodium phosphate, potassium phosphate, di-sodium hydrogen phosphate dihydrate, or sodium dihydrogen phosphate dihydrate) at a concentration of about 1 to about 25 mM, or at a concentration of about 0.1-1.0 mg/mL; (iv) a fourth buffering agent (e.g., sodium chloride, potassium chloride, sodium phosphate, potassium phosphate, di-sodium hydrogen phosphate dihydrate, or sodium dihydrogen phosphate dihydrate) at a concentration of about 1 to about 25 mM, or at a concentration of about 0.1-1.0 mg/mL; (v) one or more stabilizers (e.g., alanine, valine, leucine, serine, threonine, lysine histidine, tryptophan, aspartic acid, or glutamic acid) at a concentration of about 0.1 to about 3.0 mg/mL; and (vi) one or more surfactants (e.g., non-ionic surfactants like a polysorbate (e.g., polysorbate 80 or polysorbate 20) or nonoxynols; anionic surfactants like docusate; or cationic surfactants like quaternary ammonium salts) at a concentration of about 0.05% (v/v) to about 2.5% (v/v), or at a concentration of about 0.1 to about 3.0 mg/mL. In some embodiments, the stabilizer may be an amino acid, and in some embodiments, the surfactant may be a non-ionic surfactant such as a polysorbate. For the purposes of the present disclosure, it should be understood that QM1114-drug product (also referred to herein as “QM1114-DP” or simply “QM1114”) may exemplify any of the foregoing embodiments, or any of the following embodiments.

The liquid composition (e.g., QM1114) may have a pH between 5.5 and 8. In some embodiments, the pH is between 6.0 and 7.5, for example about 6.3, 6.35, 6.4, 6.45, 6.5, 6.55, 6.6, 6.65, 6.7, 6.75, 6.8, 6.85, 6.9, 6.95, 7.0, 7.05, 7.1, 7.15, 7.2, 7.25, 7.3, 7.35, 7.4, 7.45 or 7.5. In some embodiments, the pH is between 6.6 and 6.9. The liquid composition preferably comprises an aqueous diluent, more preferably water, for example sterile water, water for injection, purified water, and sterile water for injection.

The liquid composition may comprise (e.g. in addition to BoNT) the following components to provide a liquid formulation of BoNT that is stable when stored:
a. a source of chloride ions selected from NaCL at concentration of 100-300 mM, and KCl at a concentration of 1-25 mM;
b. more than one source of phosphate ions selected from sodium phosphate, potassium phosphate, di-sodium hydrogen phosphate, dihydrate, and sodium dihydrogen phosphate dihydrate, at a concentration of 1-50 mM;
c. a non-ionic surfactant at a concentration of 0.01-5% (v/v); and
d. an amino acid stabilizer selected from alanine, valine, leucine, isoleucine, methionine, phenylalanine, tyrosine, and tryptophan, at a concentration of 0.1-3 mg/ml.

The liquid composition according to the invention may comprise an amino acid which is tryptophan and/or tyrosine. Without willing to be bound by theory, it is hypothesized that tryptophan and/or tyrosine can prevent oxidation of the active protein which would render it nonfunctional. Indeed, it is thought that the amino acid added in molar excess over the neurotoxin will be oxidized in the first place, saving the neurotoxin. It is also hypothesized that tryptophan or tyrosine can neutralize reactive degradation products of surfactants such as polysorbates. Preferably the amino acid is tryptophan. More preferably, the amino acid is L-tryptophan.

The amino acid concentration may be from about 0.1 to 5 mg/ml, for example between 0.1 and 5 mg/ml, from 0.25 and 3 mg/ml for example about 0.25, 0.5, 1, 1.5, 2 or 3 mg/ml. Preferably, the amino acid (e.g. tryptophan and/or tyrosine) concentration is >0.5 mg/ml. For example, the amino acid concentration may have a lower limit of >0.5 mg/ml, and an upper limit of 1, 1.5, 2, 3, 4, 5, 6, 7 or 8 mg/ml. Advantageously, a concentration of >5 mg/ml may represent a de minimis concentration of amino acid, over which the stabilization effect may be particularly prevalent. See the Examples section of WO2017/203038A1, which is incorporated herein by reference.

The liquid composition may be stable over time. For example, the liquid composition may be stable for 2 months at 2 to 8° C. According to one embodiment, the liquid composition is stable for 3 months at 2 to 8° C., for example at 5° C. According to a preferred embodiment, the liquid composition is stable for 6 months at 2 to 8° C., for example at 5° C. According to one embodiment, the liquid composition is stable for 12 months at 2 to 8° C., for example at 5° C. According to one embodiment, the liquid composition is stable for 18 months at 2 to 8° C., for example at 5° C. According to one embodiment, the liquid composition is stable for 24 months at 2 to 8° C., for example at 5° C. According to one embodiment, the liquid composition is stable for 36 months at 2 to 8° C., for example at 5° C. According to one embodiment, the liquid composition is stable for 3 months at room temperature, for example at 25° C. According to one embodiment, the liquid composition is stable for 6 months at room temperature, for example at 25° C. According to one embodiment, it is stable for 2 months at 37° C.

Stability can be assessed through comparison of the activity of the botulinum neurotoxin over time. Activity of the botulinum neurotoxin may refer to the ability of the activity of the botulinum neurotoxin to bind to its target receptor on a cell, to translocate the light chain into a cell, and/or to cleave its target SNARE protein. Methods for measuring Botulinum neurotoxin activity are well known in the art. Botulinum neurotoxin activity can be assessed for example by using a mouse lethality assay (LD50) as described above, a muscle tissue based assay such as the mouse phrenic nerve hemidiaphragm assay (for example as described in Bigalke, H. and Rummel A., Toxins 7.12 (201S):489S-490S), a cell based assay (for example as described in WO201349508 or in WO2012166943) or an extracellular proteolytic activity assay such as BoTest® (Botulinum Neurotoxin Detection Kit available from BioSentinel Inc.).

Preferably, a composition according to the invention is considered stable if there is no more than a given percentage of loss of activity over a given period of time and at a given temperature.

According to one embodiment, a composition according to the invention is considered stable if there is no more than 30% loss in extracellular proteolytic activity over 3, 6, 12, 18, 24 or 36 months at 2 to 8° C., for example no more than 30% loss in extracellular proteolytic activity over 6 months at 5° C. Preferably, a composition according to the invention is considered stable if there is no more than 20% loss in extracellular proteolytic activity over 3 months at 5° C., more preferably over 6, 12, 18, 24 or 36 months at 5° C. According to another embodiment, a composition according to the invention is considered stable if there is no more than 40% loss in extracellular proteolytic activity over 3 months at room temperature, for example at 25° C. Preferably, a composition according to the invention is considered stable if there is no more than 30% loss in extracellular proteolytic activity over 3 months at 25° C., more preferably over 6 months at 25° C. According to another embodiment, a composition according to the invention is considered stable if there is no more than 50% loss in extracellular proteolytic activity over 2 months at 37° C. Extracellular proteolytic activity can be measured with the BoTest® assay.

According to one embodiment, a composition according to the invention is considered stable if there is no more than 30% loss in MLD50 units over 2, 3, 6, 12, 18, 24 or 36 months at 2 to 8° C., for example no more than 30% loss in MLD50 units over 6 months at 5° C. Preferably, a composition according to the invention is considered stable if there is no more than 20% loss in MLD50 units over 2 months at 5° C., more preferably over 3, 6, 12, 18, 24 or 36 months at 5° C. According to another embodiment, a composition according to the invention is considered stable if there is no more than 40% loss in MLD50 units over 2 or 3 months at room temperature, for example at 25° C. Preferably, a composition according to the invention is considered stable if there is no more than 30% loss in MLD50 units over 3 months at 25° C., more preferably over 6 months at 25° C. According to another embodiment, a composition according to the invention is considered stable if there is no more than 50% loss in MLD50 units over 2 months at 37° C. MLD50 units can be measured as indicated above.

The liquid composition can be suitable for injection to a patient, in particular a human patient. The quantity of botulinum neurotoxin is commonly expressed in mouse LD₅₀ (lethal dose 50) units, defined as the median lethal intraperitoneal dose in mice. The mouse LD₅₀ (MLD₅₀) unit for botulinum toxins is not a standardized unit. Indeed, assays used by different manufacturers of marketed toxins differ in particular in the choice of dilution buffer. For example the test used for BOTOX® uses saline as a diluent. It is believed that gelatine buffers protect the toxin at the high dilutions used in LD₅₀ assays. In contrast, the use of saline as a diluent is thought to lead to some loss of potency.

In some embodiments, the dilution buffer used to determine the mouse LD₅₀ is a gelatine phosphate buffer. For example, the mouse LD₅₀ can be determined as described in Hambleton, P. et al. Production, purification and toxoiding of Clostridium botulinum type A toxin. Eds. G. E. Jr Lewis, and P. S. Angel. Academic Press, Inc., New York, USA, 1981, p. 248. Briefly, botulinum toxin samples are serially diluted in 0.2% (w/v) gelatine 0.07M Na2HPO4 buffer at pH 6.5. Groups of mice (e.g., 4 to 8 mice per group) weighing about 20 g are injected intraperitoneally with a sample of diluted toxin (for example 0.5 ml per animal). Dilution groups, for example 5 dilution groups, are selected to span the 50% lethality dose. The mice are observed for up to 72 hours and the mouse lethal dose 50 (MLD₅₀) is estimated.

The liquid composition according to the invention preferably comprises from 4 to 10000 LD₅₀ units of botulinum neurotoxin per mL, more preferably from 10 to 200 LD₅₀ units of botulinum neurotoxin per mL, for example 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200 LD₅₀ units of botulinum neurotoxin per mL. The quantity of botulinum neurotoxin can also be expressed in ng.

The liquid composition according to the present invention has osmolality of 200-400 mosm/kg, and preferably from 270-310 mosm/kg, for example, 270, 275, 280, 285, 290, 295, 300, 305, or 310 mosm/kg or any value in between.

According to one embodiment, a liquid composition according to the invention may comprise or consist essentially of:

4 to 10000 LD50 units of botulinum neurotoxin per ml,

0.001 to 15% v/v polysorbate,

0.1 to 5 mg/ml tryptophan,

10 to 500 mM NaCl,

1 to 50 mM KCl,

1 to 100 mM Sodium phosphate,

and has a pH between 5.5 and 8, and is stable for 2 months (e.g. 6 months) at 5° C.

According to one embodiment, a liquid composition according to the invention may comprise or consist essentially of:
4 to 10000 LD50 units of botulinum neurotoxin per ml,
0.001 to 15% v/v polysorbate,
0.1 to 5 mg/ml tryptophan,
10 to 500 mM NaCl,
1 to 50 mM KCl,
1 to 100 mM of di-sodium hydrogen phosphate dihydrate and sodium dihydrogen phosphate dihydrate
   and has a pH between 5.5 and 8, and is stable for 2 months (e.g. 6 months) at 5° C.

According to one embodiment, a liquid composition according to the invention may comprise or consist essentially of:

10 to 2000 LD50 units of botulinum neurotoxin per ml,

0.005 to 2% v/v polysorbate,

0.1 to 5 mg/ml tryptophan,

25 to 300 mM NaCl,

1 to 10 mM KCl,

2 to 50 mM Sodium phosphate,

and has a pH between 6.0 and 7.5, and is stable for 12 months at 5° C.

According to one embodiment, a liquid composition according to the invention may comprise or consist essentially of:
10 to 2000 LD50 units of botulinum neurotoxin per ml,
0.005 to 2% v/v polysorbate,
0.1 to 5 mg/ml tryptophan,
25 to 300 mM NaCl,
1 to 10 mM KCl,
2 to 50 mM of di-sodium hydrogen phosphate dihydrate and sodium dihydrogen phosphate dihydrate,
   and has a pH between 6.0 and 7.5, and is stable for 12 months at 5° C.

According to one embodiment, a liquid composition according to the invention may comprise or consist essentially of:

10 to 2000 LD50 units of botulinum neurotoxin per ml,

0.05 to 0.2% v/v polysorbate 80,

0.1 to 5 mg/ml tryptophan,

25 to 300 mM NaCl,

1 to 10 mM KCl,

2 to 50 mM Sodium phosphate,

and has a pH between 6.0 and 7.5, and is stable for 12 months at 5° C.

According to one embodiment, a liquid composition according to the invention may comprise or consist essentially of:
10 to 2000 LD50 units of botulinum neurotoxin per ml,
0.05 to 0.2% v/v polysorbate 80,
0.1 to 5 mg/ml tryptophan,
25 to 300 mM NaCl,
1 to 10 mM KCl,
2 to 50 mM of di-sodium hydrogen phosphate dihydrate and sodium dihydrogen phosphate dihydrate,
   and has a pH between 6.0 and 7.5, and is stable for 12 months at 5° C.

According to one embodiment, a liquid composition according to the invention may comprise or consist essentially of:

Botulinum neurotoxin A,

0.2% v/v polysorbate 80,

1 mg/ml tryptophan,

140 mM NaCl,

3 mM KCl,

10 mM Sodium phosphate,

wherein the pH of said composition is approximately 6.6.

According to one embodiment, a liquid composition according to the invention may comprise or consist essentially of:
Botulinum neurotoxin A,
0.2% v/v polysorbate 80,
1 mg/ml tryptophan,
140 mM NaCl,
3 mM KCl,
10 mM of di-sodium hydrogen phosphate dihydrate and sodium dihydrogen phosphate dihydrate,
   wherein the pH of said composition is approximately 6.6.

According to one embodiment, a liquid composition according to the invention may comprise or consist essentially of:

Botulinum neurotoxin A,

0.04% v/v polysorbate 80,

1 mg/ml tryptophan,

140 mM NaCl,

3 mM KCl,

10 mM Sodium phosphate,

wherein the pH of said composition is approximately 6.9.

According to one embodiment, a liquid composition according to the invention may comprise or consist essentially of:
Botulinum neurotoxin A,
0.04% v/v polysorbate 80,
1 mg/ml tryptophan,
140 mM NaCl,
3 mM KCl,
10 mM of di-sodium hydrogen phosphate dihydrate and sodium dihydrogen phosphate dihydrate,
   wherein the pH of said composition is approximately 6.9.

According to one embodiment, a liquid composition according to the invention may comprise or consist essentially of:

Botulinum neurotoxin B,

0.25% v/v polysorbate 20,

4 mg/ml tryptophan,

140 mM NaCl,

3 mM KCl,

10 mM Sodium phosphate,

wherein the pH of said composition is approximately 7.4.

According to one embodiment, a liquid composition according to the invention may comprise or consist essentially of:
Botulinum neurotoxin B,
0.25% v/v polysorbate 20,
4 mg/ml tryptophan,
140 mM NaCl,
3 mM KCl,
10 mM of di-sodium hydrogen phosphate dihydrate and sodium dihydrogen phosphate dihydrate,
   wherein the pH of said composition is approximately 7.4.

According to one embodiment, a liquid composition according to the invention may comprise or consist essentially of:

Botulinum neurotoxin A,

0.01% v/v polysorbate 80,

0.25 mg/ml tryptophan,

255 mM NaCl,

2 mM Sodium phosphate,

wherein the pH of said composition is approximately 7.2.

According to one embodiment, a liquid composition according to the invention may comprise or consist essentially of:

Botulinum neurotoxin A,

0.01% v/v polysorbate 80,

0.25 mg/ml tryptophan

255 mM NaCl,

10 mM KCl,

50 mM Sodium phosphate,

wherein the pH of said composition is approximately 6.3.

According to one embodiment, a liquid composition according to the invention may comprise or consist essentially of:

Botulinum neurotoxin A,

1% v/v polysorbate 80,

0.25 mg/ml tryptophan,

255 mM NaCl,

50 mM Sodium phosphate,

wherein the pH of said composition is approximately 6.3.

According to one embodiment, a liquid composition according to the invention may comprise or consist essentially of:

Botulinum neurotoxin A,

1% v/v polysorbate 80,

3 mg/ml tryptophan,

255 mM NaCl,

10 mM KCl,

50 mM Sodium phosphate,

wherein the pH of said composition is approximately 7.2.

According to one embodiment, a liquid composition according to the invention may comprise or consist essentially of:
Botulinum neurotoxin A,
1% v/v polysorbate 80,
3 mg/ml tryptophan,
255 mM NaCl,
10 mM KCl,
50 mM di-sodium hydrogen phosphate dihydrate and sodium dihydrogen phosphate dihydrate,
   wherein the pH of said composition is approximately 7.2.

According to one embodiment, a liquid composition according to the invention may comprise or consist essentially of:

Botulinum neurotoxin A,

0.1% v/v polysorbate 80,

1.625 mg/ml tryptophan,

140 mM NaCl,

3 mM KCl,

10 mM Sodium phosphate,

wherein the pH of said composition is approximately 6.75.

According to one embodiment, a liquid composition according to the invention may comprise or consist essentially of:
Botulinum neurotoxin A,
0.1% v/v polysorbate 80,
1.625 mg/ml tryptophan,
140 mM NaCl,
3 mM KCl,
10 mM di-sodium hydrogen phosphate dihydrate and sodium dihydrogen phosphate dihydrate,
   wherein the pH of said composition is approximately 6.75.

According to one embodiment, a liquid composition according to the invention may comprise or consist essentially of:

Botulinum neurotoxin A,

0.01% v/v polysorbate 80,

1 mg/ml tryptophan,

140 mM NaCl,

3 mM KCl,

10 mM Sodium phosphate,

wherein the pH of said composition is approximately 6.75.

According to one embodiment, a liquid composition according to the invention may comprise or consist essentially of:
Botulinum neurotoxin A,
0.01% v/v polysorbate 80,
1 mg/ml tryptophan,
140 mM NaCl,
3 mM KCl,
10 mM di-sodium hydrogen phosphate dihydrate and sodium dihydrogen phosphate dihydrate,
   wherein the pH of said composition is approximately 6.75.

According to one embodiment, a liquid composition according to the invention may comprise or consist essentially of:

Botulinum neurotoxin A,

0.1% v/v polysorbate 80,

1 mg/ml tryptophan,

140 mM NaCl,

3 mM KCl,

10 mM Sodium phosphate,

wherein the pH of said composition is approximately 6.75.

According to one embodiment, a liquid composition according to the invention may comprise or consist essentially of:
Botulinum neurotoxin A,
0.1% v/v polysorbate 80,
1 mg/ml tryptophan,
140 mM NaCl,
3 mM KCl,
10 mM di-sodium hydrogen phosphate dihydrate and sodium dihydrogen phosphate dihydrate,
   wherein the pH of said composition is approximately 6.75.

According to one embodiment, a liquid composition according to the invention may comprise or consist essentially of:

Botulinum neurotoxin A,

1% v/v polysorbate 80,

1 mg/ml tryptophan,

140 mM NaCl,

3 mM KCl,

10 mM Sodium phosphate,

wherein the pH of said composition is approximately 6.75.

According to one embodiment, a liquid composition according to the invention may comprise or consist essentially of:
Botulinum neurotoxin A,
1% v/v polysorbate 80,
1 mg/ml tryptophan,
140 mM NaCl,
3 mM KCl,
10 mM di-sodium hydrogen phosphate dihydrate and sodium dihydrogen phosphate dihydrate,
   wherein the pH of said composition is approximately 6.75.

According to one embodiment, a liquid composition according to the invention may comprise or consist essentially of:

Botulinum neurotoxin B,

15% v/v polysorbate 20,

1 mg/ml tryptophan,

140 mM NaCl,

3 mM KCl,

10 mM Sodium phosphate,

wherein the pH of said composition is approximately 7.4.

According to one embodiment, a liquid composition according to the invention may comprise or consist essentially of:
Botulinum neurotoxin B,
15% v/v polysorbate 20,
1 mg/ml tryptophan,
140 mM NaCl,
3 mM KCl,
10 mM di-sodium hydrogen phosphate dihydrate and sodium dihydrogen phosphate dihydrate,
   wherein the pH of said composition is approximately 7.4.

According to one embodiment, a liquid composition according to the invention may comprise or consist essentially of:

Botulinum neurotoxin B,

15% v/v polysorbate 20,

4 mg/ml tryptophan,

140 mM NaCl,

3 mM KCl,

10 mM Sodium phosphate,

wherein the pH of said composition is approximately 7.4.

According to one embodiment, a liquid composition according to the invention may comprise or consist essentially of:
Botulinum neurotoxin B,
15% v/v polysorbate 20,
4 mg/ml tryptophan,
140 mM NaCl,
3 mM KCl,
10 mM di-sodium hydrogen phosphate dihydrate and sodium dihydrogen phosphate dihydrate,
   wherein the pH of said composition is approximately 7.4.

According to one embodiment, a liquid composition according to the invention may comprise or consist essentially of:

Botulinum neurotoxin B,

0.25% v/v polysorbate 20,

4 mg/ml tryptophan,

140 mM NaCl,

3 mM KCl,

10 mM Sodium phosphate,

wherein the pH of said composition is approximately 7.4.

According to one embodiment, a liquid composition according to the invention may comprise or consist essentially of:
Botulinum neurotoxin B,
0.25% v/v polysorbate 20,
4 mg/ml tryptophan,
140 mM NaCl,
3 mM KCl,
10 mM di-sodium hydrogen phosphate dihydrate and sodium dihydrogen phosphate dihydrate,
   wherein the pH of said composition is approximately 7.4.

Enzymatic activity of BoNT can also be determined by BOTEST™ (e.g., BOTEST™ Matrix A Botulinum Neurotoxin Detection Kit; Cat. A1015 from BioSentinel), which is an assay that provides detection of BoNT proteolytic activity in an optimized reaction buffer. Intended uses of BOTEST™ include quantification and characterization of BoNT pharmaceutical preparations, drug discovery, high throughput screening applications, and detection applications. BOTEST™ contains a reporter that consists of residues 141-206 of SNAP-25, the naturally occurring substrate of BoNT/A and E. The substrate is sandwiched between two fluorescent proteins, a cyan fluorescent protein (CFP) derivative, and a yellow fluorescent protein (YFP) derivative. The CFP and YFP moieties form a donor-acceptor Forster Resonance Energy Transfer (FRET) pair. The use of a FRET donor-acceptor pair separated by the substrate for BoNT allows for the detection of BoNT proteolytic activity. In the absence of BoNT, the reporter is intact and the CFP and YFP moieties are in close proximity. Excitation of CFP results in energy transfer to YFP due to FRET. As a consequence, CFP emission is quenched while YFP emits fluorescence due to FRET. In the presence of BoNT, the reporter is cleaved by the proteolytic activity of BoNT. The CFP and YFP moieties are physically separated and FRET can no longer occur. CFP emission is restored and YFP emission is reduced. Thus, BOTEST™ utilizes fluorescent reporters that give a ratiometric response when specifically cleaved by BoNT, which provides output as relative fluorescent units (RFU) or an emissions ratio. These outputs can be used to calculate botulinum units (“BU”, which may be synonymous with enzymatic unit “U” or international units “IU”)/ml or BU/concentration of BoNT (e.g., mg). In other words, enzymatic activity of BoNT can also be determined by a first cell-based assay e.g. that provides detection of BoNT proteolytic activity in an optimized reaction buffer. Such first cell-based assay contains a reporter that consists of residues 141-206 of SNAP-25, the naturally occurring substrate of BoNT/A and E. The substrate is sandwiched between two fluorescent proteins, a cyan fluorescent protein (CFP) derivative, and a yellow fluorescent protein (YFP) derivative. The CFP and YFP moieties form a donor-acceptor Forster Resonance Energy Transfer (FRET) pair. The use of a FRET donor-acceptor pair separated by the substrate for BoNT allows for the detection of BoNT proteolytic activity. In the absence of BoNT, the reporter is intact and the CFP and YFP moieties are in close proximity. Excitation of CFP results in energy transfer to YFP due to FRET. As a consequence, CFP emission is quenched while YFP emits fluorescence due to FRET. In the presence of BoNT, the reporter is cleaved by the proteolytic activity of BoNT. The CFP and YFP moieties are physically separated and FRET can no longer occur. CFP emission is restored and YFP emission is reduced. Thus, first cell-based assay utilizes fluorescent reporters that give a ratiometric response when specifically cleaved by BoNT, which provides output as relative fluorescent units (RFU) or an emissions ratio. These outputs can be used to calculate botulinum units (“BU”, which may be synonymous with enzymatic unit “U” or international units “IU”)/ml or BU/concentration of BoNT (e.g., mg).

In some embodiments, the activity or specific activity of a formulation may be established using a BOTEST™ Matrix A Botulinum Neurotoxin Detection Kit (Cat. A1015 from BioSentinel). The BOTEST™ Matrix A Botulinum Neurotoxin (BoNT) Detection Kit is an in vitro assay for the detection and quantification of BoNT serotype A (BoNT/A) in complex matrices such as serum, blood, food, water, bacterial supernatant, and pharmaceutical samples. The BOTEST™ Matrix A Kit consists of two primary reagents—the Matrix A Beads and the BOTEST™ A/E Reporter. The Matrix A Beads capture, concentrate, and isolate BoNT/A complex or holotoxin out of complex matrices from samples ranging in size from 100 μl to 50 ml. The BOTEST™ A/E Reporter then detects the amount of captured BoNT/A providing assessments of BoNT/A activity and quantity. Depending on the matrix composition and sample size, picomolar sensitivities can be achieved in <3 hours and femtomolar sensitivities in 24 hours. The Matrix A Beads consist of a proprietary anti-BoNT/A antibody covalently conjugated to magnetic beads. These beads are added to a sample containing BoNT/A and incubated to allow BoNT/A binding. Interfering compounds that might otherwise impede BoNT/A activity determinations are removed by washing the Matrix A Beads. The BOTEST™ A/E Reporter is modeled after the naturally occurring substrates of proteolytic BoNT and detects the endopeptidase activity of BoNT/A. The BoTest™ A/E Reporter contains amino acids 141-206 of SNAP-25, encompassing both the exosite binding sites and the cleavage site of BoNT/A. The reporter has a high affinity for BoNT/A and, when incubated with BoNT/A bound to the Matrix A Beads, rapidly and sensitively detects BoNT/A proteolytic activity. A description of one suitable protocol for this kit is provided in the Examples section below. In other words, the activity or specific activity of a formulation may be established using a second cell-based assay e.g. for the detection and quantification of BoNT serotype A (BoNT/A) in complex matrices such as serum, blood, food, water, bacterial supernatant, and pharmaceutical samples. The second cell-based assay consists of two primary reagents—the Beads (consisting of anti-BoNT/A antibody covalently conjugated to magnetic beads) and the Reporter which is a polypeptide modeled after the naturally occurring substrates of proteolytic BoNT and detects the endopeptidase activity of BoNT/A. The Beads capture, concentrate, and isolate BoNT/A complex or holotoxin out of complex matrices from samples ranging in size from 100 μl to 50 ml. The Reporter then detects the amount of captured BoNT/A providing assessments of BoNT/A activity and quantity. Depending on the matrix composition and sample size, picomolar sensitivities can be achieved in <3 hours and femtomolar sensitivities in 24 hours. As mentioned above, the Beads consist of anti-BoNT/A antibody covalently conjugated to magnetic beads. These beads are added to a sample containing BoNT/A and incubated to allow BoNT/A binding. Interfering compounds that might otherwise impede BoNT/A activity determinations are removed by washing the Beads. The Reporter is modeled after the naturally occurring substrates of proteolytic BoNT and detects the endopeptidase activity of BoNT/A. The Reporter contains amino acids 141-206 of SNAP-25, encompassing both the exosite binding sites and the cleavage site of BoNT/A. The reporter has a high affinity for BoNT/A and, when incubated with BoNT/A bound to the Beads, rapidly and sensitively detects BoNT/A proteolytic activity. A description of one suitable protocol for this kit is provided in the Examples section below.

For the purposes of the present disclosure, a liquid composition (e.g., QM1114) that is suitable for the disclosed treatment may have an enzymatic activity of at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, or at least 115 BU/ml as determined by a BOTEST™ (in other words, as determined by the first cell-based assay), as described above and in the examples section. In some embodiments, a liquid composition (e.g., QM1114) that is suitable for the disclosed treatment may have an enzymatic activity of about 95, about 96, about 97, about 98, about 99, about 100, about 101, about 102, about 103, about 104, about 105, about 106, about 107, about 108, about 109, about 110, about 111, about 112, about 113, about 114, or about 115 BU/ml as determined by a BOTEST™ (in other words, as determined by the first cell-based assay). In some embodiments, a liquid composition (e.g., QM1114) that is suitable for the disclosed treatment may have an enzymatic activity of 95, 96, 97, 98, 99, 100, 101, 102, 103, 104, 105, 106, 107, 108, 109, 110, 111, 112, 113, 114, or 115 BU/ml as determined by a BOTEST™ (in other words, as determined by the first cell-based assay). In some embodiments, a liquid composition (e.g., QM1114) that is suitable for the disclosed treatment may have an enzymatic activity of about 95, about 96, about 97, about 98, about 99, about 100, about 101, about 102, about 103, about 104, about 105, about 106, about 107, about 108, about 109, about 110, about 111, about 112, about 113, about 114, or about 115 BU/ml as determined by a BOTEST™ (in other words, as determined by the first cell-based assay). In some embodiments, a liquid composition (e.g., QM1114) that is suitable for the disclosed treatment may have an enzymatic activity of 95-100, 96-100, 97-100, 98-100, 99-100, 96-99, 97-99, 98-99, or 97-98 BU/ml as determined by a BOTEST™ (in other words, as determined by the first cell-based assay).

In some aspects, the enzymatic activity may be normalized by the concentration of the BoNT in a given formulation to take into account the relative level of activity of different BoNT enzymes (i.e., the calculation of specific activity). For example, specific activity or a normalized activity may be calculated by dividing the results of a BOTEST™ (e.g., BOTEST™ Matrix A Botulinum Neurotoxin Detection Kit; Cat. A1015 from BioSentinel) with the concentration or amount of BoNT in the formulation being tested. For example, specific activity or a normalized activity may be calculated by dividing the results of the first or second cell-based assay with the concentration or amount of BoNT in the formulation being tested. In some embodiments, a liquid composition (e.g., QM1114) that is suitable for the disclosed treatment may have an enzymatic activity normalized by BoNT concentration (i.e., specific activity) of at least 0.98, at least 0.99, at least 1.00, at least 1.01, at least 1.02, at least 1.03, at least 1.04, at least 1.05, at least 1.06, at least 1.07, at least 1.08, at least 1.09, at least 1.10, at least 1.11, at least 1.12, at least 1.13, at least 1.14, at least 1.15, at least 1.16, at least 1.17, at least 1.18, at least 1.19, or at least 1.20 BU/RBU of BoNT. In some embodiments, a liquid composition (e.g., QM1114) that is suitable for the disclosed treatment may have an enzymatic activity normalized by BoNT concentration of about 0.98, about 0.99, about 1.00, about 1.01, about 1.02, about 1.03, about 1.04, about 1.05, about 1.06, about 1.07, about 1.08, about 1.09, about 1.10, about 1.11, about 1.12, about 1.13, about 1.14, about 1.15, about 1.16, about 1.17, about 1.18, about 1.19, or about 1.20 BU/RBU of BoNT. In some embodiments, a liquid composition (e.g., QM1114) that is suitable for the disclosed treatment may have an enzymatic activity normalized by BoNT concentration of 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, or 1.20 BU/RBU of BoNT. Additionally or alternatively, in some embodiment, a liquid composition (e.g., QM1114) that is suitable for the disclosed treatment may have an enzymatic activity normalized by BoNT concentration (i.e., specific activity) of at least 0.12, at least 0.13, at least 0.14, at least 0.15, at least 0.16, at least 0.17, at least 0.18, at least 0.19, at least 0.20, at least 0.21, at least 0.22, at least 0.23, at least 0.24, or at least 0.25 BU/pg of BoNT. In some embodiments, a liquid composition (e.g., QM1114) that is suitable for the disclosed treatment may have an enzymatic activity normalized by BoNT concentration of about 0.12, about 0.13, about 0.14, about 0.15, about 0.16, about 0.17, about 0.18, about 0.19, about 0.20, about 0.21, about 0.22, about 0.23, about 0.24, or about 0.25 BU/pg of BoNT. In some embodiments, a liquid composition (e.g., QM1114) that is suitable for the disclosed treatment may have an enzymatic activity normalized by BoNT concentration of 0.12, 0.13, 0.14, 0.15, 0.16, 0.17, 0.18, 0.19, 0.20, 0.21, 0.22, 0.23, 0.24, or 0.25 BU/pg of BoNT. For the purposes of the normalizing activity by concentration, the activity can be determined by a BOTEST™ and divided by concentration of BoNT, which can be determined using, for example, a BoNT-specific ELISA (e.g., a BOLISA®).

A BOLISA®, or botulinum neurotoxin sandwich ELISA, can be used to detect and quantify total BoNT mass from liquid matrices using a traditional sandwich ELISA approach. Several different BOLISA® kits are available for various serotypes (e.g., BoNT/A Cat. A1029; BoNT/B Cat. A1045; BoNT/B4 Cat. A1048; BoNT/C Cat. A1042; BoNT/E Cat. A1034) from BioSentinel. In the BOLISA® kits (in other words, in a BoNT-specific ELISA), an anti-BoNT antibody is bound to a solid support, and a sample containing BoNT is contacted with the support. BoNT that is present in the sample is bound by the antibody, and all other materials are removed or washed away. A second detectably-labeled antibody that binds the same serotype of BoNT is added and the amount of BoNT in the sample can be determined by detecting or quantifying the output of the detectable label (e.g., fluorescence, enzymatic activity, colorometric change, etc.). For the purposes of calculating specific activity (i.e., level of activity in a formulation divided by concentration of BoNT in the formulation), the BOLISA® assay (in other words, a BoNT-specific ELISA) is particularly useful. The output of the BOLISA® assay (in other words, a BoNT-specific ELISA) can be weight/ml (e.g., mg, μg, or pg/ml) or relative BOLISA® units (RBU)/ml

Relative potency or enzymatic activity can also be determined using cell-based potency assays (CBPAs). CBPAs can evaluate the key steps of BoNT action: receptor binding, internalization-translocation, and catalytic activity; and therefore can be used in place of the standard mouse bioassay described above. For example, one particularly useful CBPA is described in PCT/IB2021/056210 (WO2022/009182) and comprises (a) distributing about two different BoNT samples to about two containers comprising cells expressing a SNAP25 protein, wherein the first BoNT sample is a reference sample of a known potency and the second BoNT sample is a test sample of unknown potency, (b) incubating the cells with the BoNT for a period of time, (c) determining the ratio of cleaved SNAP25 protein to uncleaved SNAP25 protein corresponding to the reference sample and the test sample, and (d) identifying the potency of the test sample relative to the reference sample. Optionally, a third BoNT sample, a quality control sample, of known potency, can be distributed to a third container and utilized as a positive control. In some embodiments, (c) may comprise subjecting the cleaved and uncleaved SNAP25 proteins to Western blot and densitometric quantification. In some embodiments, the period of time is for about 6, 12, 16, 20, 24, 32, 40, 48, or 56 hours. In some embodiments, the about two containers each comprise a plurality of wells. In some embodiments, the about two different BoNT samples are serially diluted across the plurality of wells. In some aspects, the about two containers are tissue culture plates. In some embodiments, the about two containers are 48-, 96-, 384-, or 1536-well plates. In some embodiments, the cells are adhered or attached to the about two containers. In some embodiments, the cells natively express SNAP25. In some embodiments, the cells express a heterologous SNAP25. In some embodiments, the cells are non-neuronal cells. In some embodiments, the cells are genetically modified. In some embodiments, the cells are neuronal cells. In some embodiments, the neuronal cells are motor neurons. In some embodiments, the cells are treated with a non-proliferation agent. In some embodiments, the non-proliferation agent inhibits γ-secretase. In some aspects, the non-proliferation agent is DAPT. In some embodiments, a protease inhibitor is added to the about two containers upon conclusion of (b). In some embodiments, the cells are lysed after incubating the cells with the BoNT. In some embodiments, the cells are lysed by sonication. In some embodiments, the cells are lysed by addition of a lysis agent. In some embodiments, the lysis agent comprises a detergent. This assay provides the same potency data as the current animal-based potency assays without having to rely on LD₅₀ experimentation on mice.

Another potential CBPA can utilize human induced pluripotent stem cells (hiPSC) derived from motor neurons and SNAP25 cleavage detected by Western blot. Another example is a CBPA utilizing differentiated human neuroblastoma SiMa cells and a sandwich ELISA that measures BoNT/A-dependent intracellular increase of cleaved SNAP25 can be used to determine potency. Once such exemplary assay is disclose, for example, in Salas et al., Botulinum neurotoxin serotype A specific cell-based potency assay to replace the mouse bioassay, PLoS ONE, 2012, 7(11):e49516.

In some embodiments, the relative potency of a liquid composition (e.g., QM1114) that is suitable for the disclosed treatment may be about 115, about 116, about 117, about 118, about 119, about 120, about 121, about 122, about 123, about 124, about 125, about 126, about 127, about 128, about 129, about 130, about 131, about 132, about 133, about 134, about 135, about 136, about 137, about 138, about 139, or about 140 CBpA units as determined by a cell-based assay (e.g., a hiPSC-based assay or a SiMa-cell based assay, as discussed above). In some embodiments, the relative potency of a liquid composition (e.g., QM1114) that is suitable for the disclosed treatment may be at least 115, at least 116, at least 117, at least 118, at least 119, at least 120, at least 121, at least 122, at least 123, at least 124, at least 125 at least 126, at least 127, at least 128, at least 129, at least 130, at least 131, at least 132, at least 133, at least 134, at least 135, at least 136, at least 137, at least 138, at least 139, or at least 140 CBpA units as determined by a cell-based ELISA. In some embodiments, the relative potency of a liquid composition (e.g., QM1114) that is suitable for the disclosed treatment may be 115, 116, 117, 118, 119, 120, 121, 122, 123, 124, 125, 126, 127, 128, 129, 130, 131, 132, 133, 134, 135, 136, 137, 138, 139, or 140 units as determined by a cell-based assay (e.g., a hiPSC-based assay or a SiMa-cell based assay, as discussed above).

In some aspects, the potency or activity may be normalized the concentration of the BoNT in a given formulation to take into account the relative level of activity of different BoNT enzymes (i.e., the calculation of specific activity). For example, in some embodiments, a liquid composition (e.g., QM1114) that is suitable for the disclosed treatment may have potency normalized by BoNT concentration of at least 1.20, at least 1.21, at least 1.22, at least 1.23, at least 1.24, at least 1.25, at least 1.26, at least 1.27, at least 1.28, at least 1.29, at least 1.30, at least 1.31, at least 1.32, at least 1.33, at least 1.34, at least 1.35, at least 1.36, at least 1.37, at least 1.38, at least 1.39, or at least 1.40 CBpA units/concentration of BoNT (e.g., mg). In some embodiments, a liquid composition (e.g., QM1114) that is suitable for the disclosed treatment may have potency normalized by BoNT concentration of about 1.20, about 1.21, about 1.22, about 1.23, about 1.24, about 1.25, about 1.26, about 1.27, about 1.28, about 1.29, about 1.30, about 1.31, about 1.32, about 1.33, about 1.34, about 1.35, about 1.36, about 1.37, about 1.38, about 1.39, or about 1.40 CBpA units/concentration of BoNT (e.g., mg). In some embodiments, a liquid composition (e.g., QM1114) that is suitable for the disclosed treatment may have potency normalized by BoNT concentration of 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, or 1.40 CBpA units/concentration of BoNT (e.g., mg). For the purposes of the normalizing potency/activity by concentration, the potency or activity can be determined by a cell-based assay (e.g., the cell-based assay disclosed in PCT/IB2021/056210, as discussed above) and the concentration of the of the formulation can be determined by, for example, an ELISA assay (e.g., BOLISA®).

### III. Treatment

Provided herein are methods of treating moderate to severe glabellar lines and/or lateral canthal lines in a human subject, comprising administering a therapeutically effective amount of a liquid composition (e.g., QM1114 or a “ready-to-use liquid composition) comprising a botulinum neurotoxin to the subject, thereby reducing the appearance of moderate to severe glabellar lines. The liquid composition may correspond to any of the foregoing embodiments disclosed in Section II supra.

In addition to utilizing a “ready-to-use” liquid composition (e.g., QM1114) that does not require reconstitution or admixing, the disclosed treatments are further distinguishable over prior BoNT-based treatments in that the liquid composition comprises a BoNT (e.g. BoNT/A) that is more enzymatically active than competing products (e.g., XEOMIN®, BOTOX®) when normalized by the concentration of BoNT in the formulation. The “competing products” described herein may be referred to as BoNT formulations that are stored in a dry form (e.g. lyophilized powder form) and require reconstitution for administration to a subject. As a result, the liquid composition (e.g., QM1114) can include less BoNT than competing products, which makes it generally safer and less likely to cause adverse events or side effects, such as eyelid ptosis. Accordingly, the methods and uses disclosed herein may comprise a step of selecting a subject that is at risk of developing eyelid ptosis, has previously suffered from eyelid ptosis, or has previously been treated e.g., for GL, LCL, or a combination thereof) with a BoNT formulation and experienced eyelid ptosis. In some embodiments, the methods and uses disclosed herein may comprise a step of selecting a subject that has not previously been treated (e.g., for GL, LCL, or a combination thereof) with a BoNT formulation.

The higher level of enzymatic activity in the liquid composition (e.g., QM1114) also results in a higher relative potency (when normalized by BoNT concentration) compared to competing products (e.g., XEOMIN®, BOTOX®). Higher activity and higher potency allows the disclosed methods to provide a more rapid onset of results in terms of reduction of GL and LCL, longer sustained results, and lower occurrence of side effects (e.g. ptosis) compared to competing products (e.g., XEOMIN®, BOTOX®).

Accordingly, the present disclosure provides methods of treating GL and LCL with BoNT, such as QM1114, that are safer and/or result in fewer or less severe side effects relative to competing products (e.g., BOTOX COSMETIC®, XEOMIN®, and JEUVEAU®). The present disclosure also provides methods of treating GL and LCL with BoNT, such as QM1114, that are less likely to cause eyelid ptosis than competing products (e.g., BOTOX COSMETIC®, XEOMIN®, and JEUVEAU®). The present disclosure also provides methods of treating GL and LCL with BoNT, such as QM1114, that provide more rapid onset of therapeutic effect than competing products (e.g., BOTOX COSMETIC®, XEOMIN®, and JEUVEAU®). The present disclosure also provides methods of treating GL and LCL with BoNT, such as QM1114, that provide a longer duration of therapeutic effect than competing products (e.g., BOTOX COSMETIC®, XEOMIN®, and JEUVEAU®).

Additionally or alternatively, the disclosure provides methods of treating glabellar lines (GL), lateral canthal lines (LCL), or a combination thereof with a reduced risk of eyelid ptosis in a human subject in need thereof, comprising administering a therapeutically effective amount of a liquid composition comprising a botulinum neurotoxin to the subject, thereby reducing the appearance of the GL, LCL, or combination thereof, wherein the botulinum neurotoxin an enzymatic activity normalized by concentration of botulinum neurotoxin of at least 1.16 BU/BoNT. The human subject may be at risk or considered likely of developing eyelid ptosis upon the administration of botulinum neurotoxin (e.g., a botulinum neurotoxin formulation with an enzymatic activity normalized by concentration of botulinum neurotoxin of less than 1.16 BU/BoNT or a specific activity of less than about 2.0×10⁸ U/mg, such as Botox Cosmetic®, Xeomin® and Jeuveau®). The human subject may also be suffering from eyelid ptosis and/or have a history of developing eyelid ptosis upon the administration of botulinum neurotoxin. In another embodiment, the human subject is not one that (i) has no at risk of developing eyelid ptosis, (ii) is not suffering from eyelid ptosis and/or, (iii) has no history of developing eyelid ptosis upon the administration of botulinum neurotoxin at any dose or enzymatic activity.

Specifically with respect to eyelid ptosis, the present disclosure provides evidence of eyelid ptosis rates with QM1114 that are significantly lower than those observed with other competing products. For example, in a review of studies with over 8000 patients in total, the rate of blepharoptosis was found to be about 2.5%. Specifically, eyelid ptosis incidences have been observed for Botox (3%), and Jeuveau (2%). As shown in Example 1, the eyelid ptosis rate observed when treating LCL/GL with QM1114 was as low as 0.9% in some studies. Accordingly, in some embodiments of the disclosed treatments (for LCL, GL, or a combination thereof) the rate of eyelid ptosis may be less than 2%, less than 1.9%, less than 1.8%, less than 1.7%, less than 1.5%, less than 1.4%, less than 1.3%, less than 1.2%, less than 1.1%, less than 1.0%, or as low as 0.9%. In some embodiments of the disclosed treatments (for LCL, GL, or a combination thereof) the rate of eyelid ptosis may be about 2%, about 1.9%, about 1.8%, about 1.7%, about 1.5%, about 1.4%, about 1.3%, about 1.2%, about 1.1%, about 1.0%, or about 0.9%. In some embodiments of the disclosed treatments (for LCL, GL, or a combination thereof) the rate of eyelid ptosis may be 1.9%, 1.8%, 1.7%, 1.5%, 1.4%, 1.3%, 1.2%, 1.1%, 1.0%, or 0.9% or less.

Further, in some embodiments, the present disclosure provides methods of treating lateral canthal lines (LCL) in a subject comprising administering a liquid composition of BoNT (e.g., QM1114), wherein the treatment is higher than the response rate of treating LCL with BOTOX COSMETIC® (in other words, a BoNT formulation that is stored in a dry form (e.g. lyophilized powder form) and requires reconstitution for administration to a subject).

In some embodiments, between 1 and 100 units of botulinum toxin is administered to the subject. In some embodiments, between 10 and 75 units of botulinum toxin is administered to the subject. In some embodiments, between 25 and 75 units of botulinum toxin is administered to the subject. In some embodiments, 10, 15, 20, 25, 30, 35, 40, 45, 50, 55, 60, 65, 70, 75, 80, 85, 90, 95, or 100 units of botulinum toxin is administered to the subject. In some embodiments, between 50 and 250 units of botulinum toxin is administered to the subject. In some embodiments, between 75 and 200 units of botulinum toxin is administered to the subject. In some embodiments, the concentration of the liquid composition being administered to the subject is between 1 and 300 units of botulinum toxin/mL, such as 1, 5, 10, 15, 20, 25, 30, 35, 40, 45, 50, 55, 60, 65, 70, 75, 80, 85, 90, 95, 100, 110, 115, 120, 125, 130, 135, 140, 145, 150, 155, 160, 165, 170, 175, 180, 185, 190, 195, 200, 210, 215, 220, 225, 230, 235, 240, 245, 250, 255, 260, 265, 270, 275, 280, 285, 290, 295, or 300 units/mL.

In some embodiments, the composition is administered by injection. In some embodiments, the injection is subdermal, transdermal, intradermal or intramuscular. In some embodiments, the method comprises multiple injections in the glabellar region. In some embodiments, the sites of adjacent injections are separated by about 0.5 to 10 cm. In some embodiments, the sites of adjacent injections are separated by about 1.5 to 3 cm. In some embodiments, the injection can be unilateral. In some embodiments, the injection can be bilateral.

In some embodiments, the injections are in the procerus muscle and the corrugator supercillii muscles on each side of the face, and in some embodiments, the injections are performed in a particular order, for example, starting in the procerus muscle followed by corrugator supercillii muscles on each side of the face, moving outwards from the median. In some embodiments, all the injections are about 1 cm above the upper orbital rim and internal to the mid-pupillary lines.

In some embodiments, all the injections are at least 1 cm above the central eyebrow or the bony supraorbital ridge.

In some embodiments, the composition may be administered to treat, prevent, or improve lateral canthal lines (LCL). In some embodiments, the treatment may include about three injections (one injection per injection site). For example, the treatment may include 1, 2, 3, 4, or 5 injections. The position of the injections may be adjusted in accordance with the LCL pattern of rhytids for the individual subject. Depending on the pattern of rhytids for individual subjects, if the lines in the LCL region were above and below the lateral canthus, injections were administered as described in FIG. 12A, for examples. Alternatively, if the lines in the LCL for the individual subject were primarily below the lateral canthus, injections were administered as described in FIG. 12B, for example. In some embodiments, the injection points may be at the external part of the orbicularis oculi and, when applicable, at about 1-2 cm from the orbital rim. Some embodiments may comprise three injections of equal volume (100 μl) administered to each side of the face (i.e., six injections total).

In some embodiments, the composition may be administered to treat, prevent, or improve GL and LCL concurrently (i.e., at the same time). In some embodiments, treatment may include, for example, 11 injections (one injection per injection site) of equal volume (100 μl) administered to the GL area (five injections) and LCL area (three injections to each side of the face). In some embodiments, the concurrent treatment of GL and LCL may comprise 5, 6, 7, 8, 9, 10, 11, 12, 13, or 14 or more injections. For example, for GL, the five injection sites may include two injections in each corrugator supercilii muscle, and one injection in the procerus muscle, and for LCL, the position of the injections may be adjusted in accordance with the LCL pattern of rhytids for the individual subject. Depending on the pattern of rhytids for individual subjects, if the lines in the LCL region were above and below the lateral canthus, injections may be administered as described in FIG. 12A. Alternatively, if the lines in the LCL for the individual subject were primarily below the lateral canthus, injections may be administered as described in FIG. 12B. In some embodiments, the injection points for treating LCL may be at the external part of the orbicularis oculi and, when applicable, at about 1-2 cm from the orbital rim.

In some embodiments, the method is repeated at intervals from about 3 months to about 6 months to inhibit said recurrence. In some embodiments, the method is repeated at intervals of about 4 months to inhibit said recurrence.

The method provided herein results in a temporary reduction in the appearance of glabellar lines and/or lateral canthal lines in the subject. The efficacy of the treatment can be assessed by methods known to those skilled in the art. The exemplary assessment methods are provided below and in the Examples.

a. 4-Point Photographic Scale of Glabellar Line Severity: Investigator Live Assessment (GL-ILA)

The validated 4-point Photographic Scale of Glabellar Line Severity includes two grading systems: one for Investigator live assessments at maximum frown, and one for Investigator live assessments at rest. The scale represents the severity of glabellar lines from none (grade 0), mild (grade 1), moderate (grade 2) to severe glabellar lines (grade 3). Each grade is also depicted by an individual photograph and a descriptive text. The Investigators will be trained on the use of the 4-point Photographic Scale. The Investigators will use the 4-point Photographic Scale for direct, live comparison with the subject's face at screening, baseline (prior to treatment), and at all post-treatment visits. Subjects will make their assessment of glabellar line severity independently of the Investigator's assessment. Subjects will be asked to evaluate their glabellar lines at maximum frown at screening, baseline (prior to treatment), and at all post-treatment visits using the Static 4-Point Categorical Scale.

| Grade | Severity of Glabellar Lines | Description |
|---|---|---|
| 0 | No wrinkles | Smooth skin |
| 1 | Mild wrinkles | Fairly smooth skin |
| 2 | Moderate wrinkles | Frown lines |
| 3 | Severe wrinkles | Deep frown lines |

b. Global Aesthetic Improvement Scale (GAIS)

Subjects will rate the global aesthetic improvement of their glabellar lines at maximum frown, relative to their pre-treatment appearance, using the following categorical scale at all post-treatment visits.

Rating
Very Much Improved
Much Improved
Improved
No Change
Worse
Much Worse
Very Much Worse

Subjects will be asked: “How would you rate the change in appearance of your glabellar lines (lines between your eyebrows) at maximum frown compared with immediately before the injection?”

Subjects will be instructed to select the one rating that best describes the degree to which the appearance of their glabellar lines at maximum frown have changed relative to baseline. The subject may review the baseline photograph to aid in the assessment.

c. Diary Card

Subjects will be asked to record their assessment of study treatment response in a diary card starting the day after treatment (Day 1) through day 7 (study visit 3). They will be asked to respond “yes” or “no” to the following question: “Since being injected have you noticed an improvement in the appearance of your glabellar lines (lines between your eyebrows)?” Subjects are to complete the diary card daily and return the diary to study center at the Day 7 visit.

d. FACE-Q

The FACE-Q is a patient-reported outcome instrument to evaluate the experience and outcomes of aesthetic facial procedures from the subject's perspective. FACE-Q is composed of over 40 scales, covering four domains (Satisfaction with Facial Appearance, Health Related Quality of Life, Adverse Effects, and Process of Care). Each domain has one or more independently functioning scale(s). For the purpose of this study and given the condition treated, the Psychological Function scale has been selected to be completed by the subjects at the time points indicated in the Schedule of Assessments.

e. Facial Lines Treatment Satisfaction Questionnaire

These questions ask the subject to think about the area of the face that was treated with their most recent procedure. The subjects select a response that best matches how much they agree with a given statement. An exemplary questionnaire is provided below. Only one response for each question.

| | Thinking about the area of my face that was treated | Strongly | | | Strongly |
|---|---|---|---|---|---|
| | with my most recent procedure . . . | disagree | Disagree | Agree | agree |
| 1 | I would have this treatment done again | 1 | 2 | 3 | 4 |
| 2 | I would recommend this treatment to others | 1 | 2 | 3 | 4 |
| 3 | Treatment results are what I expected | 1 | 2 | 3 | 4 |
| 4 | I feel like this is the right treatment choice for me | 1 | 2 | 3 | 4 |
| 5 | I am happy with the results of my treatment | 1 | 2 | 3 | 4 |
| 6 | I feel comfortable with how my face looks when I laugh | 1 | 2 | 3 | 4 |
| 7 | I feel comfortable with how my face looks when I smile | 1 | 2 | 3 | 4 |
| 8 | My face expresses my emotions | 1 | 2 | 3 | 4 |
| 9 | I feel comfortable with how my face looks when I | 1 | 2 | 3 | 4 |
| | express emotions | | | | |
| 10 | My facial lines aren't very visible | 1 | 2 | 3 | 4 |
| 11 | My facial lines are minimized | 1 | 2 | 3 | 4 |
| 12 | I look youthful | 1 | 2 | 3 | 4 |
| 13 | I have a natural look | 1 | 2 | 3 | 4 |
| 14 | I loch great for my age | 1 | 2 | 3 | 4 |
| 15 | I look beautiful | 1 | 2 | 3 | 4 |
| 16 | I look relaxed | 1 | 2 | 3 | 4 |
| 17 | I look attractive | 1 | 2 | 3 | 4 |
| 18 | I look well-rested | 1 | 2 | 3 | 4 |
| 19 | I look renewed | 1 | 2 | 3 | 4 |

Overall, thinking about the area of my face that was treated with my most recent procedure . . .

| | | Strongly | | | Strongly |
|---|---|---|---|---|---|
| | | disagree | Disagree | Agree | agree |
| 20 | I am satisfied with the outcome of my treatment | 1 | 2 | 3 | 4 |
| 21 | I am satisfied with the improvement in my facial lines | 1 | 2 | 3 | 4 |

f. Satisfaction with Lines (SWL) Questionnaire

At baseline (prior to treatment) and at all post-treatment visits, subjects will be asked to complete the validated SWL Questionnaire, developed by Galderma.

g. Independent Photographic Reviewer (IPR)

Three IPRs, blinded to the subject's randomized treatment, will perform photographic assessments of each subject's GL at maximum frown using the validated 4-point Photographic Scale of Glabellar Line Severity. IPR assessment is performed at the end of the study for all subjects. The IPRs will use the scale for comparison with photographs of each subject's GL at maximum frown at baseline and each post-treatment visit. The IPR score is determined as the median of the scores of the three reviewers. The IPRs are not involved in any other aspect of the study.

### EXAMPLES

### Example 1: Efficacy and Safety of QM1114-DP for the Treatment of Moderate to Severe Glabellar Lines (Study 43QM1602)

A multi-center. Randomized, double-blind, placebo-controlled study to evaluate the efficacy and safety of QM1114-drug product (DP) for the treatment of moderate to severe glabellar lines was conducted on 300 subjects in 10 sites in the US and Canada. QM1114-DP is exemplary of a liquid composition of botulinum neurotoxin (e.g. BoNT/A) of the invention comprising the at least 4 buffering agents outlined in the description above. Thus, reference to QM1114-DP throughout these examples refers to an exemplary “liquid composition comprising a botulinum neurotoxin” of the invention. Group 1 (the treatment group) consisted of 225 subjects who received a single treatment of QM1114-DP (5 units) at baseline. Group 2 (the placebo group) consisted of 75 subjects who received a single placebo treatment at baseline. The efficacy and safety of the treatment were measured in follow up visits at 7 days, 14 days and at months 1, 2, 3, 4, 5 and 6 after administration.

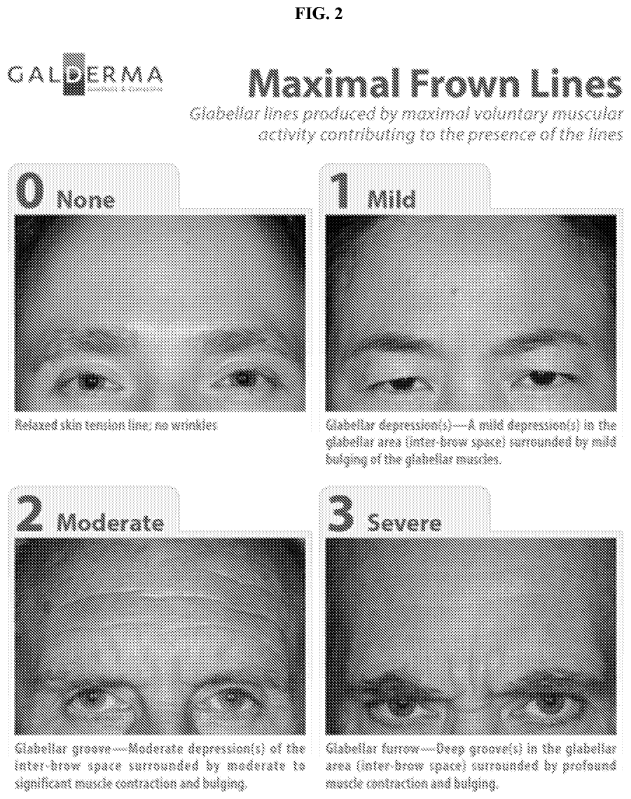

Primary efficacy endpoint was measured using a “composite responder rate” for glabellar lines. A composite responder rate for glabellar lines is evaluated using two assessment scales: an investigator live assessment (ILA) 4-point photographic scale and subject self-assessment (SSA) static 4-point categorical scale at maximum frown at Month 1. A “composite responder” refers to a subject who achieves a score of 0 or 1 in glabellar line severity and at least 2 grades improvement from baseline on both ULA and SSA scales concurrently. FIG. 2 shows the glabellar line severity scoring matrix.

Primary efficacy endpoint for this study showed that 82.9% of subjects in the treatment group (165/199) were composite responders whereas no subject in the placebo group (0%-0/67) was a composite responder. Primary efficacy endpoint measured at Month 1, composite rate, showed high statistically significant results (p<0.001) compared to Placebo.

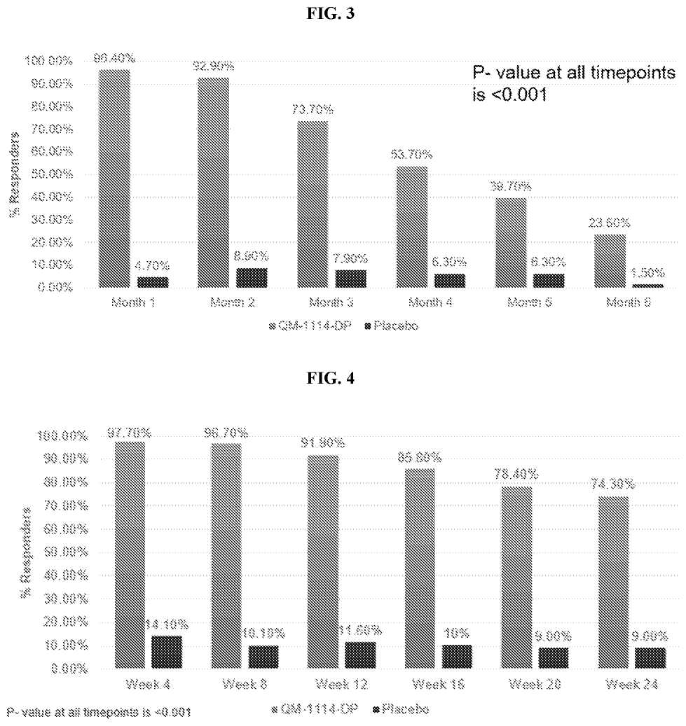

For secondary efficacy analysis, ILA responder rate over time was measured. FIG. 3 shows the results of the ILA responder rate over time. At every time point, the treatment group showed a significantly higher ILA response rate as compared to the placebo group (p<0.001). See FIG. 3.

Next, an exploratory efficacy analysis was conducted using a Global Aesthetic Improvement Score (GAIS) analysis on the subjects. In this analysis, a responder is a subject who responds “Improved,” “Much improved,” or “Very much improved” on the subject GAIS. In this instance, Subject responder rates on GAIS were high (majority assessed themselves as improved or better through 6 months). See FIG. 4. Subjects in the treatment group reported onset of effects as early as Day 0. The median response onset in the treatment group was 2 days.

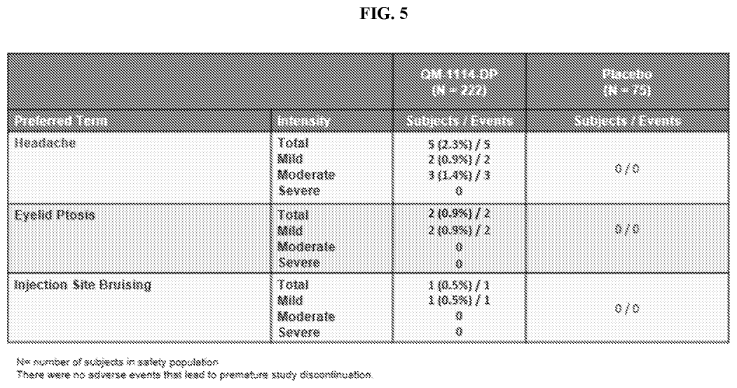

Safety: Overall incidence of adverse events (AEs) were comparable to placebo. All serious adverse events (SAEs) were assessed as unrelated to the study drug or injection procedure. All treatment-emergent adverse events (TEAEs—also “related adverse events”) were mild or moderate in intensity, the most frequent TEAEs were headache, eyelid ptosis and bruising. See FIG. 5. No subject died or experienced a TEAE resulting in premature study discontinuation.

### Conclusions

Efficacy: QM-1114-DP showed statistically significant difference against placebo in primary endpoint and secondary endpoint in reducing glabellar lines in subjects.

Onset: The treatment group subjects reported seeing beneficial effects as soon as Day 0; and the Median of Onset was 2 days.

Safety: Overall safety of QM-1114-DP is acceptable with a favorable risk/benefit profile. The treatment group showed very low eyelid ptosis rate (0.9%).

### Example 2: Efficacy and Safety of QM1114-DP for the Treatment of Moderate to Severe Lateral Canthal Lines (Study 43QM1901)

A multi-center. Randomized, double-blind, placebo-controlled study to evaluate the efficacy and safety of QM1114-DP for the treatment of moderate to severe lateral canthal lines was conducted on 300 subjects in 10 sites in the US and Canada. Group 1 (the treatment group) consisted of 225 subjects who received a single treatment of QM1114-DP (5 units) at baseline. Group 2 (the placebo group) consisted of 75 subjects who received a single placebo treatment at baseline. The efficacy and safety of the treatment were measured in follow up visits at 7 days, 14 days and at months 1, 2, 3, 4, 5 and 6 after administration.

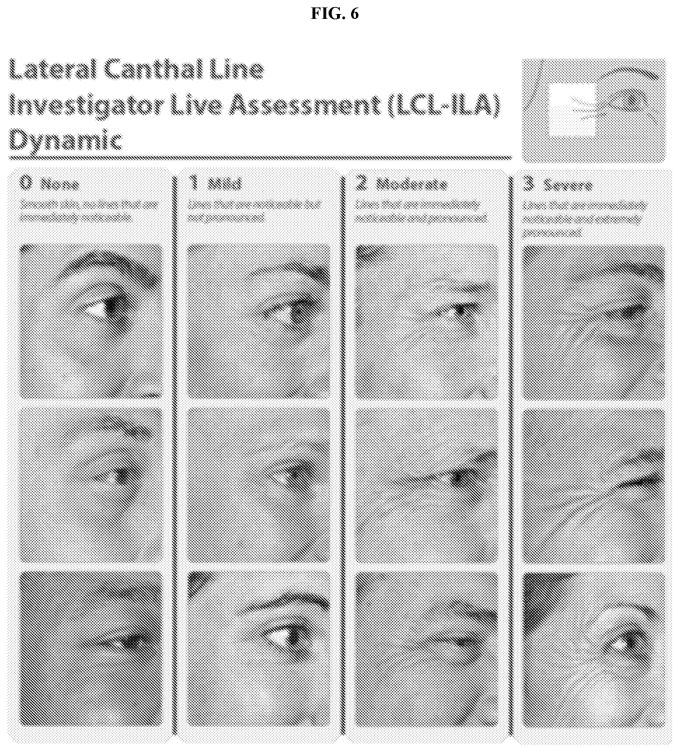

Primary efficacy endpoint was measured using a “composite responder rate” for lateral canthal lines. A composite responder rate for lateral canthal lines is evaluated using two assessment scales: an investigator live assessment (ILA) 4-point photographic scale and subject live-assessment (SLA) static 4-point categorical scale at maximum smile at Month 1. A “composite responder” refers to a subject who achieves a score of 0 or 1 in lateral canthal line severity and at least 2 grades improvement from baseline on both ULA and SSA scales concurrently. FIG. 6 shows the lateral canthal line severity scoring matrix.

Primary efficacy endpoint for this study showed that 51.8% of subjects in the treatment group (106/204) were composite responders whereas only 1.4% of subjects in the placebo group (1/69) was a composite responder. Primary efficacy endpoint measured at Month 1, composite rate, showed high statistically significant results (p<0.001) compared to Placebo.

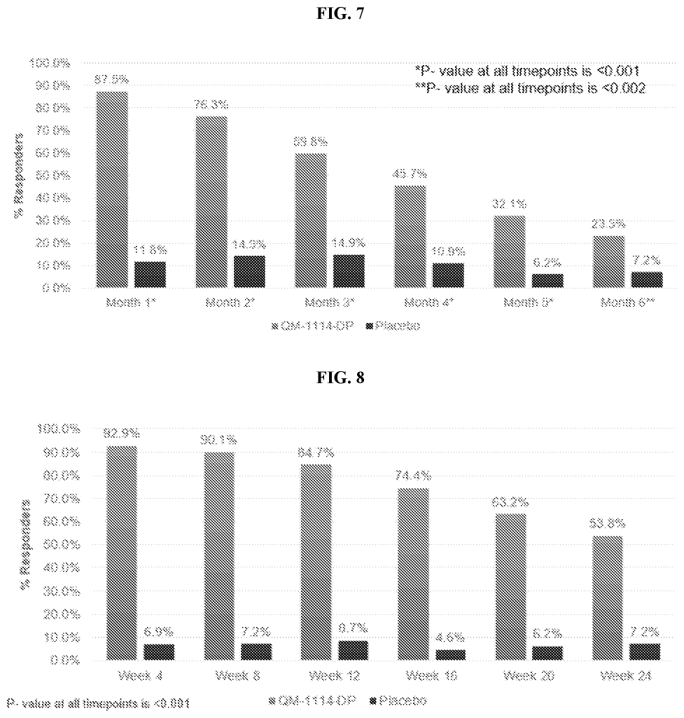

For secondary efficacy analysis, ILA responder rate over time was measured. FIG. 7 shows the results of the ILA responder rate over time. At every time point, the treatment group showed a significantly higher ILA response rate as compared to the placebo group (p<0.001). See FIG. 7.

Next, an exploratory efficacy analysis was conducted using a Global Aesthetic Improvement Score (GAIS) analysis on the subjects. In this analysis, a responder is a subject who responds “Improved,” “Much improved,” or “Very much improved” on the subject GAIS. In this instance, Subject responder rates on GAIS were high (majority assessed themselves as improved or better through 6 months). See FIG. 8. Subjects in the treatment group reported onset of effects as early as Day 0. The median response onset in the treatment group was 2 days.

Safety: Overall incidence of adverse events (AEs) were comparable to placebo. All serious adverse events (SAEs) were assessed as unrelated to the study drug or injection procedure. All treatment-emergent adverse events (TEAEs—also “related adverse events”) were mild or moderate in intensity, the most frequent TEAE was injection site bruising. See FIG. 9. No subject died or experienced a TEAE resulting in premature study discontinuation.

### Conclusions

Efficacy: QM-1114-DP showed statistically significant difference against placebo in primary endpoint and secondary endpoint in reducing lateral canthal lines in subjects.
Onset: The treatment group subjects reported seeing beneficial effects as soon as Day 0; and the Median of Onset was 2 days.

Safety: Overall safety of QM-1114-DP is acceptable with a favorable risk/benefit profile. The most common related AE was injection site bruising (4.8% in the treatment group vs. 4.1% in the placebo group).

### Example 3: Efficacy and Safety of QM1114-DP for the Treatment of Moderate to Severe Lateral Canthal Lines & Glabellar Lines (Alone or in Combination) (Study 43QM1902)

A multi-center. Randomized, double-blind, placebo-controlled study to evaluate the efficacy and safety of QM1114-DP for the treatment of moderate to severe lateral canthal lines (LCL) and glabellar lines (GL) (alone or in combination) was conducted on 413 subjects in 12 sites in the US and Canada. Group A (the LCL-alone treatment group) consisted of 118 subjects who received a single treatment of QM1114-DP (5 units) to LCL, and a single placebo treatment to GL at baseline. Group B (the GL-alone treatment group) consisted of 118 subjects who received a single treatment of QM1114-DP (5 units) to GL a single placebo treatment to LCL at baseline. Group C (the LCL/GL combination treatment group) consisted of 118 subjects who received a single treatment of QM1114-DP (5 units) to LCL, and a single treatment of QM1114-DP (5 units) to GL at baseline. Finally, Group D (the placebo group) consisted of 59 subjects who received a single treatment of placebo to LCL, and a single treatment of placebo to GL at baseline. The efficacy and safety of the treatments were measured in follow up visits at 7 days, 14 days and at months 1, 2, 3, 4, 5 and 6 after administration.

Primary efficacy endpoint for glabellar lines was measured using a “composite responder rate” for glabellar lines. A composite responder rate for glabellar lines is evaluated using two assessment scales: an investigator live assessment (ILA) 4-point photographic scale and subject self-assessment (SSA) static 4-point categorical scale at maximum frown at Month 1. A “composite responder” refers to a subject who achieves a score of 0 or 1 in glabellar line severity and at least 2 grades improvement from baseline on both ULA and SSA scales concurrently. FIG. 2 shows the glabellar line severity scoring matrix.

Primary efficacy endpoint for glabellar lines showed that 71.1% of subjects in the GL-alone treatment group (75/106) were composite responders, and 72% of subjects in the LCL/GL combination treatment group (78/108) were composite responders, whereas no subject in the placebo group (0%-0/55) was a composite responder. Primary efficacy endpoints measured at Month 1, composite rate, showed high statistically significant results (p<0.001) compared to Placebo.

Primary efficacy endpoint lateral canthal lines was measured using a “composite responder rate” for lateral canthal lines. A composite responder rate for lateral canthal lines is evaluated using two assessment scales: an investigator live assessment (ILA) 4-point photographic scale and subject live-assessment (SLA) static 4-point categorical scale at maximum smile at Month 1. A “composite responder” refers to a subject who achieves a score of 0 or 1 in lateral canthal line severity and at least 2 grades improvement from baseline on both ULA and SSA scales concurrently. FIG. 6 shows the lateral canthal line severity scoring matrix.

Primary efficacy endpoint for lateral canthal lines showed that 45.1% of subjects in the LCL-alone treatment group (53/117) were composite responders, and 55% of subjects in the LCL/GL combination treatment group (59/108) were composite responders, whereas no subject in the placebo group (0%-0/55) was a composite responder. Primary efficacy endpoints measured at Month 1, composite rate, showed high statistically significant results (p<0.001) compared to Placebo.

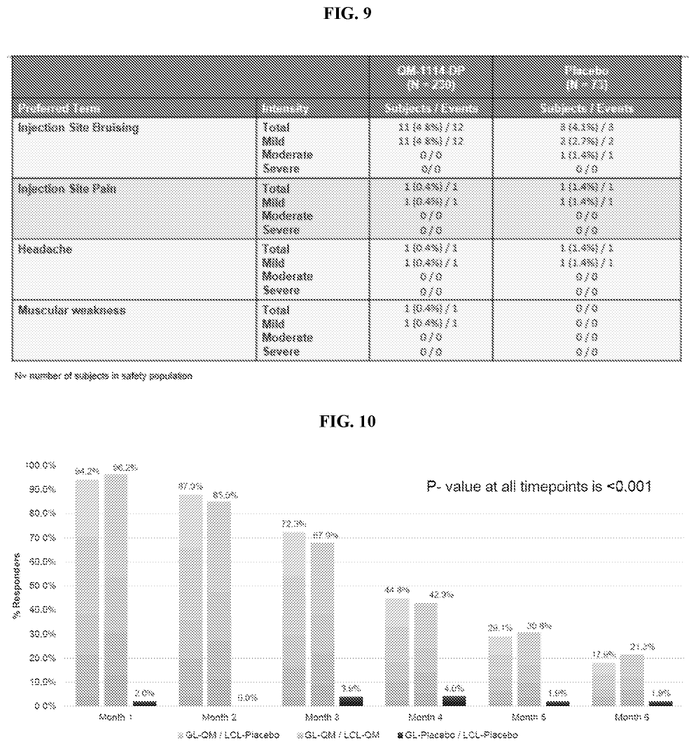

For secondary efficacy analysis for glabellar lines, ILA responder rate over time was measured. FIG. 10 shows the results of the ILA responder rate for glabellar lines over time. At every time point, the treatment groups (both GL-alone and GL/LCL combination treatment groups) showed a significantly higher ILA response rate as compared to the placebo group (p<0.001). See FIG. 10.

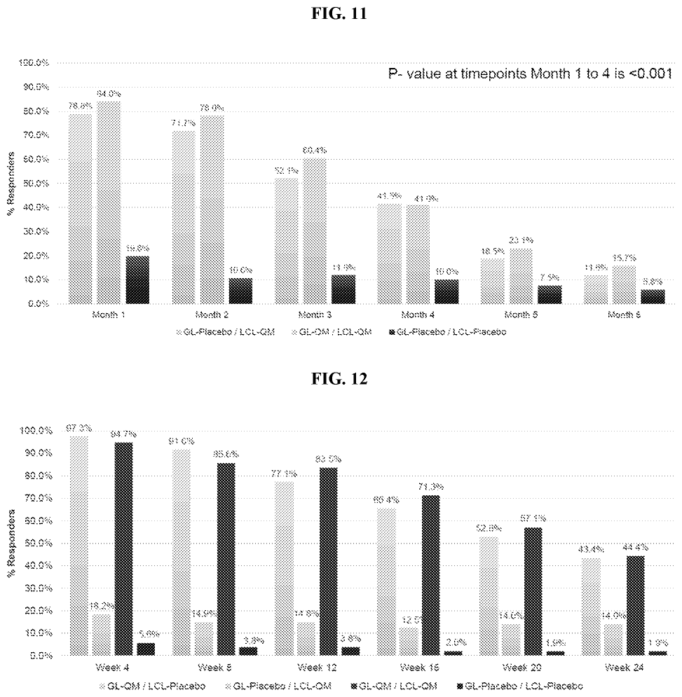

For secondary efficacy analysis for lateral canthal lines, ILA responder rate over time was measured. FIG. 11 shows the results of the ILA responder rate for lateral canthal lines over time. At every time point, the treatment groups (both LCL-alone and GL/LCL combination treatment groups) showed a significantly higher ILA response rate as compared to the placebo group (p<0.001). See FIG. 11.

Next, an exploratory efficacy analysis was conducted using a Global Aesthetic Improvement Score (GAIS) analysis on the subjects. In this analysis, a responder is a subject who responds “Improved,” “Much improved,” or “Very much improved” on the subject GAIS. In this instance, Subject responder rates for both GL and LCL on GAIS were high (majority assessed themselves as improved or better through 5 months) as compared to placebo controls. See FIG. 12. Subjects in the treatment group reported onset of effects as early as Day 0. The median response onset in the treatment group was 2-4 days.

Safety: Overall incidence of adverse events (AEs) were comparable to placebo. All serious adverse events (SAEs) were assessed as unrelated to the study drug or injection procedure. All treatment-emergent adverse events (TEAEs—also “related adverse events”) were mild or moderate in intensity, the most frequent TEAEs were headache (4.3%), injection site bruising (2.6%), and eyelid ptosis (1.7% only observed in GL-alone treatment group). No subject died or experienced a TEAE resulting in premature study discontinuation.

### Example 4: Comparing the Effects of QM-114-DP and Other Botulinum Toxins on Glabellar Lines and

In a comparison study, QM-1114-DP was compared against other available botulinum toxin products.

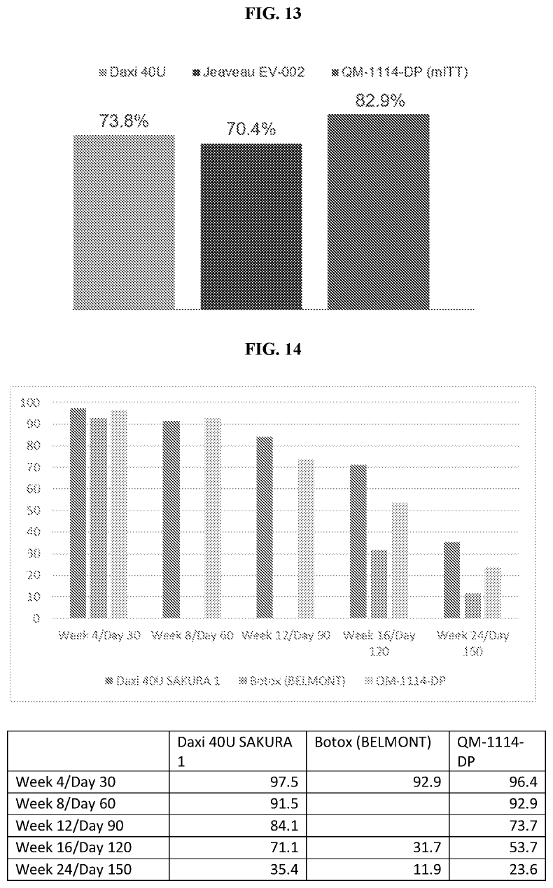

Composite 2-grade improvement for glabellar lines: QM-1114-DP showed superior composite 2-grade improvement for glabellar lines at week 4 (Day 30) as compared to Daxi 40U (DaxibotulinumtoxinA) and Jeaveau EV-002 (PrabotulinumtoxinA). Briefly, QM-1114-DP showed about 82.9% composite 2-grade improvement for glabellar lines whereas Daxi 40U showed 73.8% and Jeaveau EV-002 showed 70.4%. See FIG. 13.

Investigator Score for glabellar lines: QM-1114-DP showed comparable and lasting investigator score of none to mild for glabellar lines up to 24 weeks (150 days) when compared to Daxi 40U and Botox. See FIG. 14.

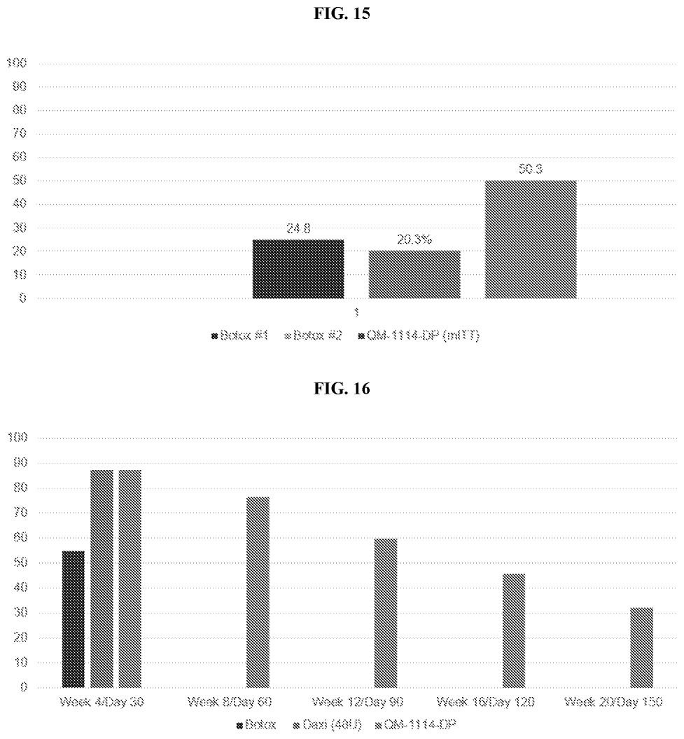

Conposite 2-grade improvement for lateral canthal lines: QM-1114-DP showed superior composite 2-grade improvement for lateral canthal lines at week 4 (Day 30) as compared to Botox. See FIG. 15.

Investigator Score for glabellar lines: QM-1114-DP showed comparable and lasting investigator score of none to mild for glabellar lines up to 24 weeks (150 days) when compared to Daxi 40U and Botox (OnabotulinumtoxinA). Interestingly, it was observed that Daxi 40U and Botox had no subjects with investigator score of none or mild after 4 weeks. See FIG. 16.

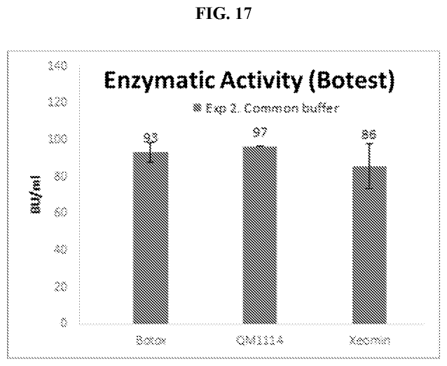

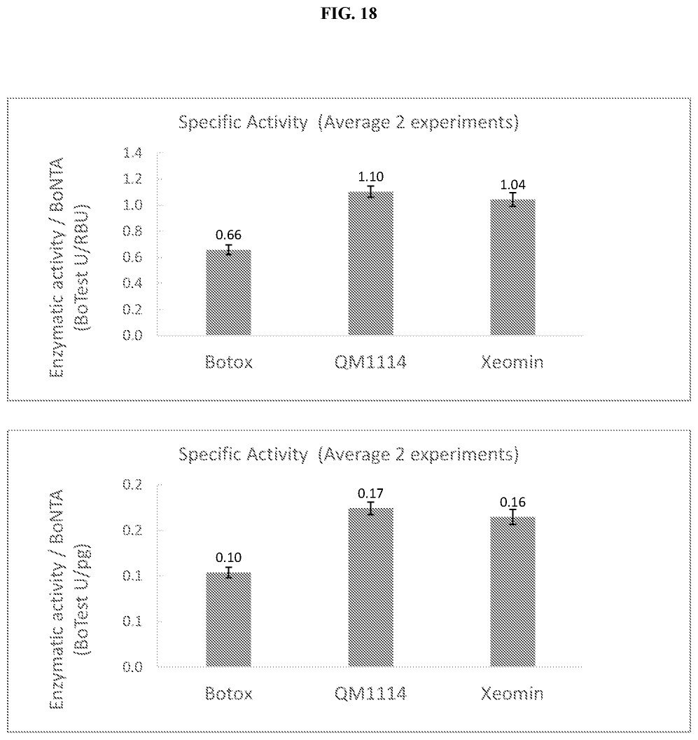

Comparing Enzymatic Activity: Enzymatic activity was measured using BoTest™ (see FIGS. 17 and 18). In the BoTest™ assay (BOTEST™ Matrix A Botulinum Neurotoxin Detection Kit; Cat. A1015 from BioSentinel), a common buffer was used for all toxins, to exclude all effects from excipients, only looking at the toxin. Briefly, samples were dispensed into microtiter wells and a Matrix Binding Buffer was added to each sample. Resuspended Matrix A beads were dispensed into each well containing a sample and the plates were incubated. The Matrix Beads were washed with Matrix Wash Buffer. The wash buffer was removed and replaced with a BoTest™ reaction buffer containing DTT. A BoTest™ Master Stock was added and then plates were covered to incubate, followed by a fluorescence reading. The observed enzymatic activity pattern looks very similar with both assays.

In FIG. 18, a normalized value (i.e., specific activity) for each product was calculated by dividing the activity (U/ml) by the amount of BoNT in the formulation, as determined using a BoNT-A-specific ELISA (BOLISA®).

Results for two repeats of the enzymatic activity experiments are shown in the tables below.

Experiment 2 (common buffer)

| | Botox | QM1114 | Xeomin |
|---|---|---|---|
| BoTEST (BoTest | 93.2 ± 11.1 | 96.5 ± 0 | 85.7 ± 24.6 |
| U/mL) | | | |
| BoLisa (RBU/ml) | 154.1 ± 9.5 | 82.9 ± 1.2 | 88.5 ± 3.1 |
| Specific activity | 0.61 | 1.16 | 0.97 |
| (BoTest U/RBU) | | | |
| Specific activity | 0.10 | 0.18 | 0.15 |
| (BoTest U/pg BoNTA) | | | |

BoTest, 3 replicates per sample (A; B, X); 2 replicates (Q). A: UD0503; B: C6463C2; Q: batch 19395; X: 030390. Analysis ocasion: 20210815
Bolisa. 3 replicates per sample (Q, X), 2 replicates (A, B). A: UD0503; B: C6463C2; Q: batch 19395; X: 030390. Analysis occasion: 20210815/A/ALS

Experiment 3 (common buffer)

| | Botox | QM1114 | Xeomin |
|---|---|---|---|
| BoTEST (BoTest | 116.9 ± 14.5 | 96.6 ± 10.9 | 108.5 ± 21.8 |
| U/mL) | | | |
| BoLisa (RBU/ml) | >165 | 92.7 ± 5.6 | 97.2 ± 4 |
| Specific activity | 0.71 | 1.04 | 1.12 |
| (BoTest U/RBU) | | | |
| Specific activity | 0.11 | 0.16 | 0.18 |
| (BoTest U/pg BoNTA) | | | |

BoTest, 4 replicates (Q, B); 5 replicates (X). B: C6463C2; Q: batch 19744; X: 034161. Analysis ocasion: 20220407/A/ALS
Bolisa. 4 replicates per sample. B: C6463C2; Q: batch 19744; X: 034161 Analysis ocassion: 20220414/A/KaVa

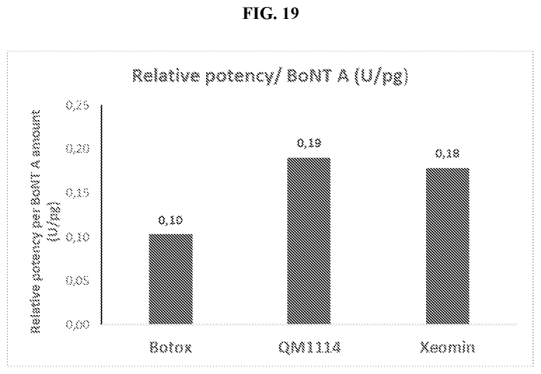

Comparing Relative Potency: The inventors have also analyzed how much toxin protein there is in QM1114-DP and competitors using a cell based assay in which hiPSC are assessed for BoNT/A-dependent intracellular increase of cleaved SNAP25, which is detected via Western blot. A common buffer was used in the CBA tests. The amount of botulinum neurotoxin type A was determined using a BoNT-A-specific ELISA (also using a common buffer). When the activity and potency are normalized (by dividing the activity and potency with the amount of toxin), it can be can seen that QM1114-DP has, the best potency per toxin molecule. Therefore, QM1114-DP is a much more potent toxin than other toxins (e.g., 90% better than Botox). See FIG. 19.

In FIG. 19 relative potency was determined using a cell based assay performed in common buffer and normalized for the protein amount of each product. The U/pg was calculated by dividing the relative potency by the amount of BoNT in the formulation, as determined using a BoNT-A-specific ELISA (BOLISA®).

### Conclusions

QM1114-DP is superior to other botulinum toxins (e.g., Daxi 40U, Jeaveau EV-002 and Botox). In particular, QM1114-DP outperformed both Xeomin and Botox in terms of potency and activity. For instance, QM1114-DP demonstrates a better responder rate for treating LCL and GL than other botulinum toxins. QM1114-DP also is less likely to cause adverse side effects such as eyelid ptosis or cause less severe eyelid ptosis than other botulinum toxins. The toxin activity per unit (IU) is higher for QM1114-DP, so one gets the same amount of treatment using less QM1114-DP, which would minimize the probability or severity of any potential adverse events. Finally, QM1114-DP has a faster onset of effect, and a longer duration of effect than other toxins tested.

### Example 5: QM1114-DP is High Purity and Complex Free

The QM-1114-DP was analyzed for protein purity using ultra-high performance liquid chromatography size exclusion chromatography (UPLC-SEC) and SDS-PAGE. For the UPLC-SEC analysis indicated that QM-1114-DP was separated on a Water BEH™ SEC column, and analyzed using absorbance (A280). SDS-PAGE was run on both reduced and unreduced material, and was stained with Colloid Coomassie to enable quantification of impurities.

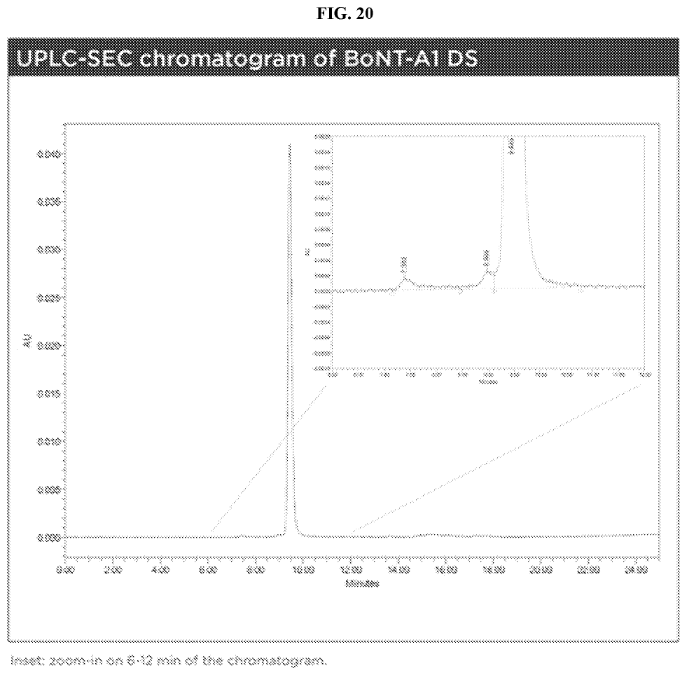

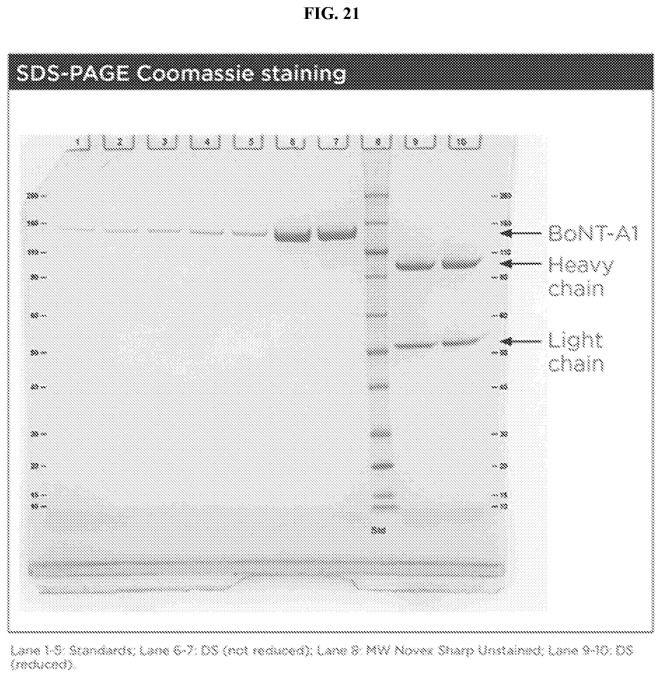

As shown in FIGS. 20 and 21, the formulation of QM-1114-DP is highly pure and free of any complexes, with BoNT-A representing the only expected bands observed in the SDS-PAGE analysis.

### Example 6: Treatment of Lateral Canthal Lines (LCL)

The QM-1114-DP was shown to have high composite≥2-grade responder rates for the treatment of glabellar lines at month 1 (83%) for up to a six month duration of effect and onset in >⅓ of subject by Day 1. Thus, a further study was undertaken to evaluate a single dose of 60 U QM-1114-DP compared with placebo for the treatment of moderate to severe LCL.

Subjects (N=303) with moderate-to-severe LCL were randomized 3:1 to QM-1114-DP 60 U or placebo. LCL severity was assessed by LCL Investigator Live Assessment (LCL-ILA) 4-Point Photographic Scale, LCL Subject Live Assessment (LCL-SLA) 4-Point Photographic Scale, and Subject-reported onset of effect, subject satisfaction. Adverse events were also tracked. The primary endpoint of the study was the composite 2-grade responder rate evaluated using the LCL-ILA and LCL-SLA at maximum smile at Month 1. A composite 2-grade responder was defined as a subject who achieved both a score of none (0) or mild (1) in LCL severity and had at least a 2-grade improvement from baseline on both the LCL-ILA and LCL-SLA Scale at maximum smile.

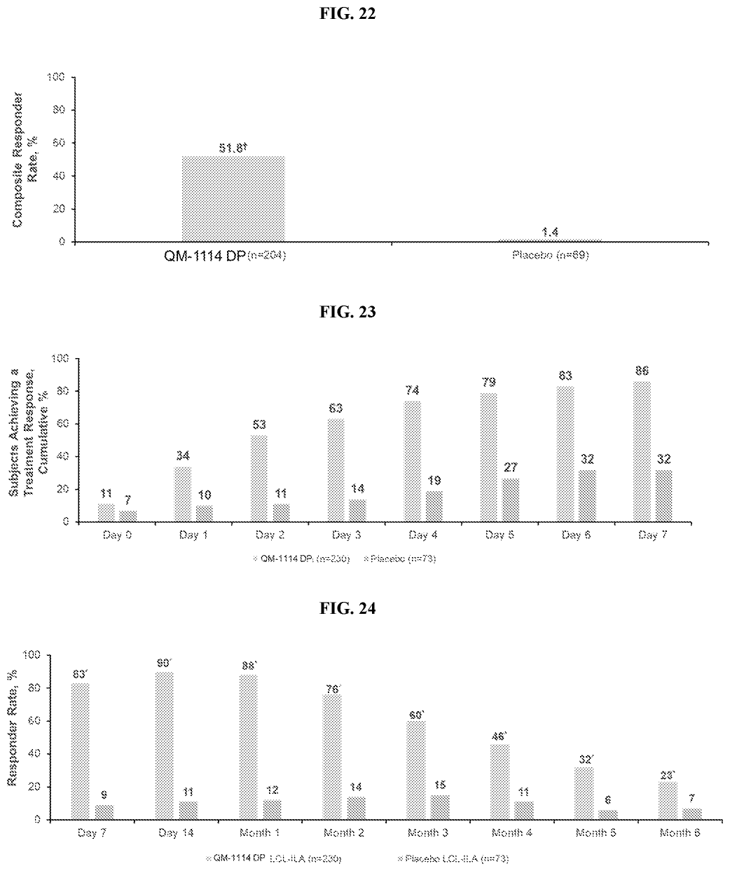

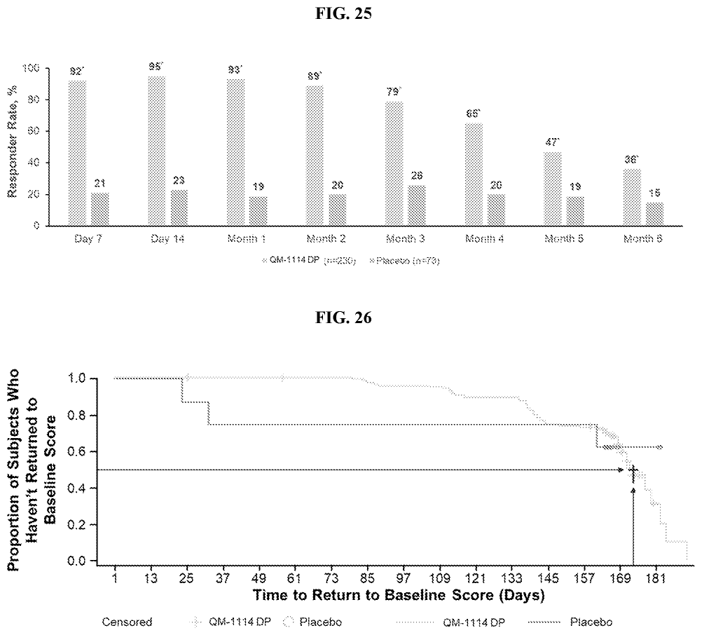

The LCL composite 2-grade responder rate was higher with relaBoNT-A vs placebo (51.8% vs 1.4%, P<0.001) (FIG. 22). The median time to onset of effect was 2 days, and 34% of subjects reported onset by Day 1 (FIG. 23). Investigators reported none or mild severity in 88% of subjects at Month 1, and effects were maintained in 23% at Month 6 (FIG. 24). Investigators reported 1-grade improvement in 93% of subjects at Month 1, and effects were maintained in 36% at Month 6 (FIG. 25).

Additionally, one exploratory endpoint was the time to return to baseline. This endpoint was assessed in subjects who achieved a score of 0 or 1 on both the LCL-ILA 4-Point Photographic Scale and LCL-SLA 4-Point Photographic Scale at maximum smile. As shown in FIG. 26, the median time to return to baseline concurrently on ILA and SLA was 173 days (24.7 weeks). Thus, QM-1114-DP provides long lasting treatment for LCL.

What is claimed:

1. A method of treating glabellar lines (GL), lateral canthal lines (LCL), or a combination thereof in a human subject with a reduced risk of eyelid ptosis, comprising administering a therapeutically effective amount of a liquid composition comprising a botulinum neurotoxin to the subject, thereby reducing the appearance of the GL, LCL, or combination thereof, wherein the botulinum neurotoxin is less likely to cause eyelid ptosis than a botulinum neurotoxin product with a lower enzymatic activity or a lower specific activity.

2. The method of claim 1, wherein the subject does not experience any serious adverse events.

3. The method of claim 1, wherein the subject is less likely to experience eyelid ptosis compared to treatment with a botulinum neurotoxin product selected from BOTOX COSMETIC®, XEOMIN®, and JEUVEAU®.

4. The method of claim 1, wherein the botulinum neurotoxin product with a lower enzymatic activity is selected from BOTOX COSMETIC®, XEOMIN®, and JEUVEAU®.

5. A method of treating glabellar lines (GL), lateral canthal lines (LCL), or a combination thereof in a human subject, comprising administering a therapeutically effective amount of a liquid composition comprising a botulinum neurotoxin to the subject, thereby reducing the appearance of the GL, LCL, or combination thereof, wherein (i) the liquid composition possesses an activity of at least about 97 BU/ml or greater.

6. The method of claim 1, wherein the liquid formulation has a specific BoNT activity of about 2.0×10⁸ U/mg total protein, wherein the specific activity (U/mg) is optionally measured using a mouse LD50 potency as U/ml divided by the total amount of protein as determined by a μBCA method (mg/ml).

7. The method of claim 1, wherein the liquid formulation has an enzymatic activity normalized by concentration of botulinum neurotoxin of at least about 1.10 BU/RBU or at least about 0.17 BU/pg, wherein the enzymatic activity of the botulinum neurotoxin is optionally determined using a BOTEST™.

8. The method of claim 1, wherein the liquid formulation has relative potency normalized by concentration of botulinum neurotoxin of at least about 1.35 CBpA units/BoNT, wherein the enzymatic activity of the botulinum neurotoxin is determined using a cell-based assay.

9. The method of claim 1, wherein the liquid formulation contains no human- or animal-derived excipients.

10. The method of claim 1, wherein the occurrence of eyelid ptosis is less than 2%, less than 1.9%, less than 1.8%, less than 1.7%, less than 1.5%, less than 1.4%, less than 1.3%, less than 1.2%, less than 1.1%, less than 1.0%, or as low as 0.9%.

11. The method of claim 1, wherein the subject does not experience eyelid ptosis.

12. The method of claim 5, wherein the subject is less likely to experience eyelid ptosis compared to treatment with a botulinum neurotoxin product selected from BOTOX COSMETIC®, XEOMIN®, and JEUVEAU®.

13. The method of claim 1, wherein the GL, LCL, or combination thereof are moderate to severe or highly severe.

14. The method of claim 1, wherein the liquid composition comprising the botulinum neurotoxin does not comprise any animal proteins or companion proteins.

15. The method of claim 1, wherein the subject has GL.

16. The method of claim 1, wherein the subject has LCL.

17. The method of claim 1, wherein the treatment provides a response rate that is higher than BOTOX COSMETIC®.

18. The method of claim 1, where the liquid composition comprises at least 4 buffering agents selected from the group consisting of sodium chloride, potassium chloride, sodium phosphate, potassium phosphate, di-sodium hydrogen phosphate dihydrate, and sodium dihydrogen phosphate dihydrate.

19. The method of claim 18, wherein the liquid composition comprises a first buffering agent present at a concentration of about 100 to about 300 mM, or at a concentration of about 0.1-10 mg/mL.

20. The method of claim 19, wherein the liquid composition comprises a second buffering agent present at a concentration of about 1 to about 25 mM, or at a concentration of about 0.1-1.0 mg/mL.

21. The method of claim 20, wherein the liquid composition comprises a third buffering agent present at a concentration of about 1 to about 25 mM, or at a concentration of about 0.1-1.0 mg/mL.

22. The method of claim 21, wherein the liquid composition comprises a fourth buffering agent present at a concentration of about 1 to about 25 mM, or at a concentration of about 0.1-1.0 mg/mL.

23. The method of claim 22, wherein the liquid composition comprises a fifth buffering agent present at a concentration of about 1 to about 25 mM, or at a concentration of about 0.1-1.0 mg/mL.

24. The method of claim 1, wherein the liquid composition comprises at least one stabilizer, which is an amino acid.

25. The method of claim 24, wherein the amino acid is selected from the group consisting of alanine, valine, leucine, isoleucine, methionine, phenylalanine, tyrosine, and tryptophan.

26. The method of claim 25, wherein the amino acid is in the D isoform or the L isoform.

27. The method of claim 1, wherein the amino acid is present at a concentration of about 0.1 to about 3.0 mg/mL.

28. The method of claim 1, wherein the liquid composition comprises at least one surfactant, which is a non-ionic surfactant.

29. The method of claim 28, wherein the non-ionic surfactant is present at a concentration of about 0.01% (v/v) to about 5.0% (v/v), or at a concentration of about 0.1 to about 3.0 mg/mL.

30. The method of claim 1, wherein the botulinum neurotoxin is selected from the group consisting of botulinum neurotoxin types A, B, C, D, E, F, and G.

31. The method of claim 30, wherein the botulinum neurotoxin is botulinum neurotoxin type A.

32. The method of claim 1, wherein the pH of the liquid composition is between 6.6 and 6.9.

33. The method of claim 1, wherein the botulinum neurotoxin has a molecular weight of about 150 kDa.

34. The method of claim 1, wherein the osmolality of the liquid composition is between 270 mosm/kg and 310 mosm/kg.

35. The method of claim 1, wherein between 1 and 100 units of botulinum toxin is administered to the subject.

36. The method of claim 38, wherein between 10 and 75 units of botulinum toxin is administered to the subject.

37. The method of claim 1, wherein the liquid composition is administered by an injection, wherein the injection is subdermal, transdermal, intradermal or intramuscular.

38. The method of claim 1, wherein the method is repeated at intervals from about 1 month to about 6 months to inhibit recurrence of GL, LCL, or a combination thereof.
